(19) United States
(12) Patent Application Publication
(10) Pub. No.: US 2022/0242963 A1
(43) Pub. Date: Aug. 4, 2022

(54) B-CELL ACTIVATING CD73 ANTIBODIES

(71) Applicant: CORVUS PHARMACEUTICALS, INC., Burlingame, CA (US)

(72) Inventors: Emily Piccione GRIFFIN, Burlingame, CA (US); Joseph BUGGY, Burlingame, CA (US); Andrew HOTSON, Burlingame, CA (US)

(21) Appl. No.: 17/290,961

(22) PCT Filed: Nov. 5, 2019
(86) PCT No.: PCT/US19/59933

§ 371 Date: May 3, 2021

Related U.S. Application Data
(60) Provisional application No. 62/756,065, filed on Nov. 5, 2018.
(60) Provisional application No. 62/848,524, filed on May 15, 2019.
(60) Provisional application No. 62/855,601, filed on May 31, 2019.

(51) Int. Cl. C07K 16/28; A61P 35/00

(52) U.S. Cl. CPC C07K 16/2896; A61K 2039/505; A61P 35/00

(57) ABSTRACT

Provided herein are, inter alia, methods and compositions using and including anti-CD73 antibodies capable of activating B cells, and affecting the redistribution of B cells from lymphoid tissues to lymphoid organs This previously unknown and unique effect of anti-CD73 antibodies may be useful for the treatment of various indications, for example, enhancing immunity to immunogenic cancers, treating autoimmune disease (e.g., multiple sclerosis), inflammatory diseases, or infectious disease.

### CROSS-REFERENCES TO RELATED APPLICATIONS

This application claims the benefit of U.S. Provisional Application No. 62/855,601, filed May 31, 2019, U.S. Provisional Application No. 62/848,524, filed May 15, 2019, and U.S. Provisional Application No. 62/756,065, filed Nov. 5, 2018, which are incorporated herein by reference in entirety and for all purposes

### REFERENCE TO A “SEQUENCE LISTING,” A TABLE, OR A COMPUTER PROGRAM LISTING APPENDIX SUBMITTED AS AN ASCII FILE

The Sequence Listing written in file 048517-543001WO_SEQUENCE_LISTING_ST25.txt, created on Oct. 31, 2019, 9,939 bytes, machine format IBM-PC, MS Windows operating system, is hereby incorporated by reference.

### BACKGROUND

The glycosyl-phosphatidylinositol-anchored CD73 antigen is considered the rate-limiting enzyme in the generation of extracellular adenosine (Stagg J, Smyth M J. Extracellular adenosine triphosphate and adenosine in cancer. Oncogene. 2010; 29:5346-58). CD73 can be found constitutively expressed at high levels on various types of cancer cells. CD73-generated adenosine is assumed to suppress adaptive anti-tumor immune responses thereby promoting tumor growth and metastasis. There is a need in the art for antibody-based CD73 cancer therapy which inhibits the catalytic activity of CD73, thereby blocking adenosine production and relieving adenosine-mediated immunosuppression. This disclosure addresses these and other needs in the art.

### BRIEF SUMMARY OF THE INVENTION

In an aspect, a method of immunostimulating a subject is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of activating B cells in a subject is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of decreasing egress of B cells from lymphoid tissue in a subject relative to a standard control is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of increasing retention of B cells in lymphoid organs in a subject relative to a standard control is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of increasing internalization of a Sphingosine-1-phosphate receptor 1 (S1PR1) in a subject relative to a standard control is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of treating a Sphingosine-1-phosphate receptor 1 (S1PR1)-associated disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of treating an infectious disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of treating an inflammatory disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, a method of treating an autoimmune disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, an anticancer immunogenic composition is provided. The composition includes an anticancer immunogenic agent and an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, an antiviral immunogenic composition is provided. The composition includes an antiviral immunogenic agent and an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

In an aspect, the disclosure provides a method of treating cancer in a patient in need thereof by administering to the patient an effective amount of an anti-CD73 antibody to effectively activate an antigen-presenting cell. In an aspect, the method comprises administering to the patient an effective amount of an anti-CD73 antibody; and monitoring a level of an antigen-presenting cell. In aspects, the method comprises administering to the patient an effective amount of an anti-CD73 antibody to effectively activate an antigen-presenting cell; and monitoring a level of the antigen-presenting cell. In aspects, monitoring the level of the antigen-presenting cell comprises obtaining a biological sample from the patient, and detecting the level of the antigen-presenting cell in the biological sample. In an aspect, the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In an aspect, the anti-CD73 antibody includes SEQ ID NO:7 (heavy chain) and SEQ ID NO:8 (light chain).

### BRIEF DESCRIPTION OF THE DRAWINGS

FIGS. 1A-1F show that CPI-006 blocks adenosine production and relieves adenosine-mediate immunosuppression. FIG. 1A provides a schematic of CD73 structure. FIG. 1B shows the CD73 catalytic activity as a function of concentration of indicated antibodies. FIG. 1C shows T-cell proliferation based on indicated antibodies. FIG. 1D shows IFN-gamma secretion based on indicated antibodies. Each symbol represents an independent donor. FIG. 1E shows the tumor volume (mm³) over the course of 20 days of treatment in an MDA-MB-231 xenograft model dosed daily with PBS, an isotype control, and 10 mg/kg CPI-006. FIG. 1F shows that CPI-006 blocks CD73 enzymatic activity in an MDA-MB-231 human triple negative breast cancer (TNBC) xenograft model, where the method for determining CD73 enzymatic activity is described by Silver et al, J. Clin Invest., 56(5):1324-1327 (1975).

FIGS. 2A-2J show that CPI-006 directly activates human B lymphocytes. FIGS. 2A-2C show that CPI-006 induces expression of B cell activation markers CD69 (FIG. 2A), CD83 (FIG. 2B), and CD25 (FIG. 2C). FIGS. 2D-2E show that CPI-006 activates antigen-presenting cells CD86 (FIG. 2D) and MHC-II (FIG. 2E). With reference to FIGS. 2A-2E, each group of four bars from left to right represent data for: untreated donors, donors treated with human IgG1 isotype control, donors treated with BCR stimulation, and donors treated with CPI-006. The experiments for FIGS. 2A-2E are based on healthy donor PBMC treated overnight with flow cytometry analysis of surface markers on B cells (CD19^{POS} CD3^{NEG}). These lymphocyte markers are consistent with activation of B cells as well as other antigen presenting cell populations. FIG. 2F shows that induction of B cell activation is unique to CPI-006. FIG. 2G shows that CPI-006 induces B cell activation via BTK as evidenced by antibody staining of CD69 (left panel) and CD83 (right panel). The bar graph legend from top to bottom is representative of the data bars from left to right. FIGS. 2H-2I show that CPI-006 induces B cell activation independent of adenosine as evidenced by antibody staining of CD69 (left panels) and CD83 (right panels). For FIG. 2H the bar graph legend from top to bottom is representative of the data bars from left to right. For FIG. 2I the legend from top to bottom is representative of the data from top to bottom. FIG. 2J shows that CPI-006 induces phosph-ERK signalizing in B cells to a greater extent than in CD4+ T cells or CD8+ T cells. For each group of four bars, the legend from top to bottom are representative of the bars from left to right.

FIGS. 3A-3D show the pharmacokinetics/pharmacodynamics and CD73 receptor occupancy of CPI-006 after a first infusion in human patients. FIG. 3A shows the serum CPI-006 concentration as function of time based on the administered dose, where the top two lines as seen at day 7 are a 12 mg/kg dose, the next lower three lines as seen at day 7 are a 6 mg/kg dose, the next lower three lines as seen at day 7 are a 3 mg/kg dose, and the bottom line with no data point at day 7 is a 1 mg/kg dose. FIG. 3B shows the percent of CD73 receptor occupancy based on dose of CPI-006 and time, where the top line at day 21 is a 12 mg/kg; the next lower line at day 21 is a 6 mg/kg dose; the next lower line at day 21 is a 3 mg/kg dose; and the lowest line at day 21 is a 1 mg/kg dose. The dotted line marks one-hundred percent occupancy CD73 receptor occupancy. FIG. 3C shows the CD73 competitive vs. non-competitive antibody at baseline (left panel) and after 24 hours (right panel). FIG. 3D shows the total cell surface CD73 and free cell surface CD73 pre-dose of CPI-006 and 30 minutes after administration of CPI-66.

FIGS. 4A-4D show that CPI-006 transiently redistributes peripheral B cells. FIG. 4A shows the level of B cells (CD19⁺CD3⁻) as a percent of total lymphocytes based on a CPI-006 dose of 1 mg/kg (left panel), 3 mg/kg (middle panel), and 6 mg/kg (right panel). FIG. 4B shows CD73 expression with a non-competing anti-CD73 antibody reported on B cells (two left panels) and T cells (two right panels). FIG. 4C shows the surface levels of CD69 (left panel) and S1P1 (right panel) on B cells treated with various doses of CPI-006. The legend from top to bottom is representative of each group of three bars from left to right. Without intending to be bound by a theory of the invention, FIG. 4D provides a model for the mechanism leading to reduction in levels of peripheral B cells by CPI-006.

FIG. 5 shows the occupancy and inhibition of CD73 in a tumor biopsy of a colorectal patient treated with 12 mg/kg of CPI-006. The tumor biopsy was of a retroperitoneal lesion obtained at trough pre-dose 3.

FIGS. 6A-6B show cancer patients being treated with various doses of CPI-006 alone (FIG. 6A) or a combination therapy of CPI-006 and ciforadenant (FIG. 6B). A cycle is once every 21 days. Disease assessment occurred every 3-4 cycles. The dashed line indicates that there was sustained CD73 receptor occupancy in peripheral blood at the 6 mg/kg and 12 mg/kg doses, but not at the 1 mg/kg and 3 mg/kg doses. HDNCK refers to head and neck cancer. mCRPC refers to metastatic castration-resistant prostate cancer. BLADD refers to bladder cancer. PANC refers to pancreatic cancer. COLORECT refers to colorectal cancer. RCC refers to renal cell carcinoma. SD indicates stable disease and PD indicates progressive disease.

FIGS. 7A-7F show that treatment with CPI-006 induces rapid changes in blood B cells and T cells. FIG. 7A shows the percentage of lymphocytes comprising CD73^{POS} B cells at pre-treatment and after 0.5 hours after administration of CPI-006 at dosages ranging from 1 mg/kg to 12 mg/kg. FIG. 7B shows the fold change in the cell frequency of CD73^{POS} CD4 T cells, CD73^{NEG} CD4 T cells, CD73^{POS} CD8 T cells, CD73^{NEG} CD8 T cells, and monocytes 0.5 hours after administration of CPI-004. FIG. 7C shows the changes in CD73^{POS} B cells over time at four CPI-006 doses. The highest line at day 10 represents 1 mg/kg, the second highest line at day 10 represents 3 mg/kg, the second lowest line at day 10 represents 6 mg/kg, and the lowest line at day 10 represents 12 mg/kg. FIG. 7D shows the changes in HLA-DR (human leukocyte antigen, DR isotype) expression in cancer patients receiving 6 mg/kg CPI-006 monotherapy. FIG. 7E shows a prostate tumor pre-treatment (left panel) and the reduction in the size of a prostate tumor after 2 cycles of treatment, i.e., after 42 days (right panel). FIG. 7F shows the changes in circulating CD73^{POS} B cells (solid line) and CD73^{NEG} B cells (dashed line) over time for the 72 year old metastatic prostate cancer patient shown in FIG. 7E, where this cancer patient had previously been treated with leuprolide/bicalutamide, abiraterone, enzalutamide and docetaxel.

FIGS. 8A-8B shows that treatment with CPI-006 induces cytokines consistent with immune activation. In particular, there is a rapid induction of inflammatory cytokines ((FIG. 8A); followed by induction of CRP (C-reactive protein) and SAA (serum amyloid A) (FIG. 8B).

FIG. 9, without intending to be bound by a theory of the mechanisms of action of CPI-006, shows a proposed model for the immunomodulatory activity of CPI-006.

FIG. 10, without intending to be bound by a theory of the mechanisms of action of CPI-006, provides a schematic of the present disclosure.

FIGS. 11A-11C show clonal abundance plots of T cell clones for three patients treated with CP1-006. Sequencing of the Vb region of the T cell receptor was performed to identify the frequency of unique T cell clones in peripheral blood samples collected pre-treatment and on-treatment at 6 weeks of 12 mg/kg CPI-006+ciforadenant (FIG. 11A), 12 mg/kg CPI-006 (FIG. 11B), or 18 mg/kg CPI-006 (FIG. 11C). Expansion of new T cell clones was observed after 6 weeks in 3 of 8 evaluated patients treated with CPI-006.

FIG. 12 shows circulating lymphocyte kinetics in the first 24 hours after treatment with CPI-006 or CPI-006 in combination with Ciforadenant (PCI-444). Whole blood was collected pre-treatment or at 0.5 hr or 24 hr after administration of CPI-006 as a single agent (left) or in combination with CPI-444 (right). The absolute number of CD19+ B cells, CD3+ T cells, CD3+CD4+ T cells, CD3+CD8+ T cells, and CD3−CD16+CD56+ NK cells were determined by flow cytometry with quantification beads. The number of circulating cells decreased at 0.5 hr post-treatment for all evaluated lymphocyte subsets but returned at 24 hr post-treatment; for B cells this was a partial return whereas other cell types returned to near baseline levels at 24 hr.

FIG. 13 shows levels of circulating CD73⁺ B cells and CD73⁻ B cells following treatment with CPI-006. Blood was collected pre-treatment or 0.5 hr after administration of CPI-006. The absolute number of CD19⁺ B cells, as well as the percentage of B cells positive for CD73, were determined using flow cytometry. The number of circulating CD73⁺ B cells substantially decreased at 0.5 hr post-treatment, whereas CD73⁻ B cells were either unchanged or modestly decreased across patients.

### DETAILED DESCRIPTION

I. Definitions

“CPI-006” or “CPX-006” is a humanized CD73 antibody, wherein the light chain is SEQ ID NO:8 and the heavy chain is SEQ ID NO:7, described herein. CPI-006 is also described in WO 2017/100670 (the disclosure of which is incorporated by reference herein in its entirety) where the heavy chain is SEQ ID NO:53, and the light chain is SEQ ID NO:55, which correspond to SEQ ID NO: 9 and SEQ ID NO: 10, respectively.

In this disclosure, “comprises,” “comprising,” “containing” and “having” and the like can have the meaning ascribed to them in U.S. patent law and can mean “ includes,” “including,” and the like. “Consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

For specific proteins (antibodies or fragments thereof) described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In aspects, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In aspects, the protein is the protein as identified by its NCBI sequence reference. In aspects, the protein is the protein as identified by its NCBI sequence reference, homolog or functional fragment thereof.

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In aspects, the amino acid side chain may be a non-natural amino acid side chain. In aspects, the amino acid side chain is H,

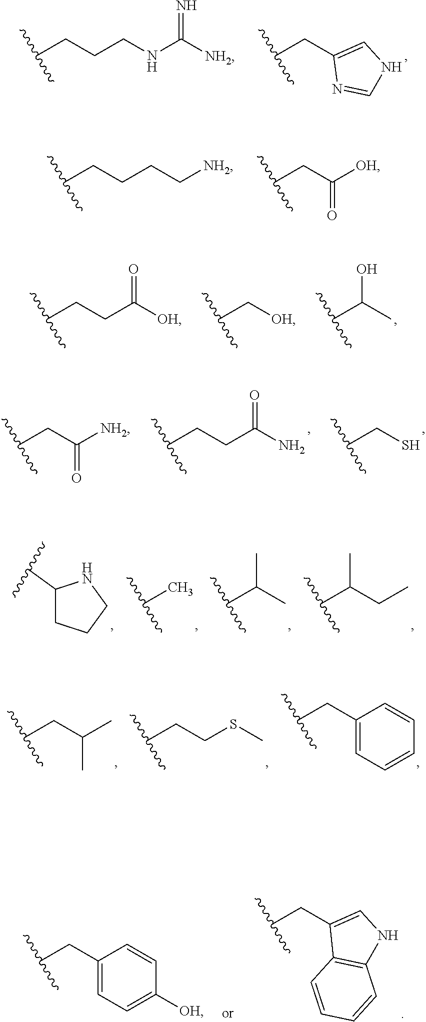

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutyric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride, cis-6-Amino-3-cyclohexene-l-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)-acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]-acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH , Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, and 4-(hydroxymethyl)-D-phenylalanine.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv , dsFv or Fab; while the terms “variable light chain,” “V_{L}” or “VL” refer to the variable region of an immunoglobulin light chain, including of an Fv, scFv , dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or using transgenic animals (Lonberg et al., WO93/12227; Kucherlapati WO91/10741, each of which is incorporated herein by reference).

Other approaches to design humanized antibodies may also be used to achieve the same result as the methods in U.S. Pat. Nos. 5,530,101 and 5,585,089 described above, for example, “superhumanization” (see Tan et al. J. Immunol. 169: 1119, 2002, and U.S. Pat. No. 6,881,557) or the method of Studnicak et al., Protein Eng. 7:805, 1994. Moreover, other approaches to produce genetically engineered, reduced-immunogenicity mAbs include “reshaping”, “hyperchimerization” and veneering/resurfacing, as described, e.g., in Vaswami et al., Annals of Allergy, Asthma and Immunology 81:105, 1998; Roguska et al. Protein Eng. 9:895, 1996; and U.S. Pat. Nos. 6,072,035 and 5,639,641.

A humanized antibody is a genetically engineered antibody in which at least one CDR (or functional fragment thereof) from a mouse antibody (“donor antibody”, which can also be rat, hamster or other non-human species) are grafted onto a human antibody (“acceptor antibody”). The human antibody is a non-natural (e.g. not naturally occurring or not naturally produced by a human) antibody that does not elicit an immune response in a human, does not elicit a significant immune response in a human, or elicits an immune response that is less than the immune response elicited in a mouse. In aspects, more than one mouse CDR is grafted (e.g. all six mouse CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, a humanized antibody may be an antibody having one or more CDRs from a donor antibody and a variable region framework (FR). The FR may form part of a constant region and/or a variable region within a human antibody. In addition, in order to retain high binding affinity, amino acids in the human acceptor sequence may be replaced by the corresponding amino acids from the donor sequence, for example where: (1) the amino acid is in a CDR; (2) the amino acid is in the human framework region (e.g. the amino acid is immediately adjacent to one of the CDR's). See, U.S. Pat. Nos. 5,530,101 and 5,585,089, incorporated herein by reference, which provide detailed instructions for construction of humanized antibodies. Although humanized antibodies often incorporate all six CDRs (e.g. as defined by Kabat, but often also including hypervariable loop H1 as defined by Chothia) from a mouse antibody, they can also be made with fewer mouse CDRs and/or less than the complete mouse CDR sequence (e.g. a functional fragment of a CDR) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

Typically a humanized antibody as provided herein may include (i) a light chain comprising at least one CDR (often three CDRs) from a mouse antibody (also referred to herein as a mouse CDR) and a human variable region framework; and (ii) a heavy chain comprising at least one CDR (often three CDRs) from the mouse antibody and a human variable region framework (FR). The light and heavy chain variable region frameworks (FRs) may each be a mature human antibody variable region framework sequence (or fragment thereof), a germline variable region framework sequence (combined with a J region sequence) (or fragment thereof), or a consensus sequence of a human antibody variable region framework sequence (or fragment thereof). In aspects, the humanized antibody includes a light chain as described in (i), a heavy chain as described in (ii) together with a light chain human constant region and a heavy chain constant region.

A “CD73 protein” or “CD73 antigen” as referred to herein includes any of the recombinant or naturally-occurring forms of the Cluster of Differentiation 73 (CD73) also known as 5′-nucleotidase (5′-NT) or ecto-5′-nucleotidase or variants or homologs thereof that maintain CD73 nucleotidase activity (e.g. within at least 50%, 80%, 90%, 95%, 96%, 97%, 98%, 99% or 100% activity compared to CD73). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD73 protein. In aspects, the CD73 protein is substantially identical to the protein identified by the UniProt reference number 21589 or a variant or homolog having substantial identity thereto. In aspects, the CD73 protein is substantially identical to the protein identified by the UniProt reference number Q61503 or a variant or homolog having substantial identity thereto.

“MEDI9447” as provided herein, refers to the anti-CD73 antibody described by Hay CM et al. (“Targeting CD73 in the tumor microenvironment with MEDI9447.” Oncoimmunology. 2016 Jul. 11; 5(8)), which is hereby incorporated in its entirety and for all purposes.

“AD2” as provided herein refers the anti-CD73 antibody described by Borrione P et al. (“CD38 stimulation lowers the activation threshold and enhances the alloreactivity of cord blood T cells by activating the phosphatidylinositol 3-kinase pathway and inducing CD73 expression.” J Immunol 162:6238-46 (1999), which is hereby incorporated in its entirety and for all purposes.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Lymphoid cell” is used in accordance with its plain ordinary meaning and refers to a subset of white blood cells responsible for immunity, and include T cells, B cells, and natural killer cells. T cells and B cells form cellular components of the adaptive immune response upon recognition of antigens. Natural killer cells are part of the innate immune system and defend the host from cancer cells and virally infected cells, for example through recognition of changes in cell surface receptors such as MHC class I.

“Dendritic cell” is used in accordance with its plain ordinary meaning and refers to an antigen-presenting cell of the immune system. Dendritic cells process and present antigens on their surfaces for recognition by T cells.

“Plasmacytoid dentric cell” (pDC) refers to a type of immune cell that links the innate and adaptive immune systems and may participate in antiviral mechanisms. For example, pDCs secrete large quantities of type 1 interferon (IFNs) in response to a viral infection. In contrast to conventional dentritic cells that leave the bone marrow as precursors, pDCs leave the bone marrow upon completion of development and go to lymphoid organs and peripheral blood.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In aspects, a control is the measurement of the activity of a protein in the absence of a compound as described herein (including embodiments and examples).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In aspects, contacting includes allowing a compound described herein to interact with a protein or enzyme that is involved in a signaling pathway.

As defined herein, the term “activation”, “activate”, “activating”, “activator” and the like in reference to a cell (e.g., B cell)-ligand interaction means positively affecting (e.g. increasing) the activity or function of the cell relative to the activity or function of the cell in the absence of the ligand. In aspects activation means positively affecting (e.g. increasing) the proliferation rate or biologic activity of the cell relative to the rate or activity of the cell in the absence of the activator. The terms may reference activation, or activating, sensitizing, or up-regulating signal transduction or enzymatic activity or gene expression of a cell. Thus, activation may include, at least in part, partially or totally increasing stimulation, increasing or enabling activation, or activating, sensitizing, or up-regulating signal transduction or enzymatic activity or gene expression relative to the absence of the activator. Activation may include, at least in part, partially or totally increasing stimulation, increasing or enabling activation, or activating, sensitizing, or up-regulating signal transduction or enzymatic activity or gene expression.

The terms “agonist,” “activator,” “upregulator,” etc. refer to a substance capable of detectably increasing the activity or proliferation of a given cell. The agonist can increase activity or proliferation by 10%, 20%, 30%, 40%, 50%, 60%, 70%, 80%, 90% or more in comparison to a control in the absence of the agonist. In aspects, proliferation or activity is 1.5-fold, 2-fold, 3-fold, 4-fold, 5-fold, 10-fold or higher than the proliferation or activity in the absence of the agonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to an agent that increases or decreases the level of a target molecule or the function of a target molecule or the physical state of the target of the molecule relative to the absence of the modulator. In aspects, the modulator increases or decreases the proliferation rate of a cell (e.g., B cell) or the function of a cell or the physical state of a cell relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia , Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, “immune deficiency” or “immunodeficiency” refers to a state in which the immune system is weak, compromised, or entirely absent Immune deficiency may result from extrinsic factors or may be intrinsic Immune deficiency may result from diseases, disorders, nutrition, or medications. Medications that may cause immune deficiency include steroids, chemotherapy, and radiation. Secondary diseases that may cause immune deficiency include AIDS, leukemia, lymphoma and viral hepatitis. Primary immunodeficiency disorders may include common variable immune deficiency and X-linked agammaglobulinemia.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease , progressive supranuclear palsy, or Tabes dorsalis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In aspects, treating is preventing. In aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In aspects, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In aspects, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound (antibody or other agent) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In aspects, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

The term “infection” or “infectious disease” refers to a disease or condition that can be caused by organisms such as a bacterium, virus, fungi or any other pathogenic microbial agents. In aspects, the infectious disease is caused by a pathogenic bacteria. Pathogenic bacteria are bacteria which cause diseases (e.g., in humans). In aspects, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In aspects, the disease is Bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e. a bacterial inflammation of the meninges); Bacterial pneumonia (i.e. a bacterial infection of the lungs); Urinary tract infection; Bacterial gastroenteritis; or Bacterial skin infections (e.g. impetigo, or cellulitis). In aspects, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

The term “CD4” as referred to herein is a glycoprotein expressed on the surface of T helper cells, regulatory T cells, monocytes, macrophages, and dendritic cells. CD4 was originally known as leu-3 and T4 (after the OKT4 monoclonal antibody). CD4 as referred to herein has four immunoglobulin domains (D₁ D₄) that are exposed on the extracellular surface of the cell, see ENTREZ No. 920, UNIPROT No. P01730, and GENBANK® Accession No. NP_000607, which are incorporated by reference.

The term “CD8” as referred to herein is a transmembrane glycoprotein that serves as a co-receptor for the T cell receptor (TCR). Like the TCR, CD8 binds to a major histocompatibility complex (MHC) molecule, but is specific for the class I MHC protein, see ENTREZ No. 925 and UNIPROT No. P01732, which are incorporated by reference herein.

The term “CD19 protein” or “CD19” as used herein includes any of the recombinant or naturally-occurring forms of B-lymphocyte antigen CD19, also known as CD19 molecule (Cluster of Differentiation 19), B-Lymphocyte Surface Antigen B4, T-Cell Surface Antigen Leu-12 and CVID3, or variants or homologs thereof that maintain CD19 activity (e.g. within at least 50%, 80%, 90%, 95%, 96%, 97%, 98%, 99% or 100% activity compared to CD19). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD19 protein. In embodiments, the CD19 protein is substantially identical to the protein identified by the UniProt reference number P15391 or a variant or homolog having substantial identity thereto.

“CD73 levels” as referred to herein is the level of CD73 expressed by a tumor.

“An elevated level of CD73” as referred to herein is an elevated level of CD73 expressed (e.g., mRNA, proteins) by a tumor in a subject when compared to a control. CD73 levels can be measured from biological samples, such as a tumor sample (e.g., resected, biopsy) or a blood sample (e.g., peripheral blood), obtained from a subject. A tumor can be a primary tumor or a metastasis. A tumor as provided herein is a cellular mass including cancer cells and non-cancer cells. The non-cancer cells forming part of a tumor may be stromal cells, and immune cells (e.g., T cells, dendritic cells, B cells, macrophages). Thus, the elevated level of CD73 may be expressed by a non-cancer cell (e.g., a stromal cell) or a cancer cell (e.g., a malignant T cell). The term is further defined herein.

An “anti-CD73 compound” refers to any compound (e.g., small molecule, peptide, protein, antibody) capable of binding to CD73 or otherwise inhibiting the ability of CD73 to perform normal functions in the adenosine pathway. In aspects, the anti-CD73 compound is an anti-CD73 antibody. Exemplary anti-CD73 antibodies include 1E9 antibodies, IgG1 antibodies, humanized 1E9 antibodies, humanized IgG1 antibodies, and the like.

“Biological sample” refers to any biological sample taken from a subject. Biological samples include blood, plasma, serum, tumors, tissue, cells, and the like. In aspects, the biological sample is a blood sample. In aspects, the biological sample is a peripheral blood sample. In aspects, the biological sample is a tumor sample. In aspects, the biological sample is a primary tumor sample. In aspects, the biological sample is a metastatic tumor sample. In aspects, the biological sample is a resected tumor sample. In aspects, the biological sample is a tumor biopsy sample. In aspects, the biological sample is a resected tumor sample from a primary tumor. In aspects, the biological sample is a resected tumor sample from a metastisic tumor. In aspects, the biological sample is a tumor biopsy sample from a primary tumor. In aspects, the biological sample is a tumor biopsy sample from a metastisic tumor. Biological samples can be taken from a subject by methods known in the art, and can be analyzed by methods known in the art.

As used herein “treating caner” and “treating a cancer tumor” means preventing an increase in size or volume of the cancer tumor. In aspects, the cancer tumor is a solid tumor. In aspects, treating a cancer tumor includes decreasing the size of volume of a cancer tumor. In aspects, treating a cancer tumor includes eliminating the cancer tumor altogether. In aspects, a cancer tumor is eliminated when it is not detectable by an imaging test such as magnetic resonance imaging (MRI), a positron emission tomography (PET) scan, X-ray computed tomography (CT), ultrasound, or single-photon emission computed tomography (SPECT). In aspects, treating a cancer tumor further comprises reducing or preventing metastasis of the cancer tumor.

Anti-CD73 Antibodies

The anti-CD73 antibodies used for the methods and included in the compositions provided herein including embodiments thereof are, inter alia, capable of binding CD73 proteins and inhibiting CD73 catalytic activity thereby preventing metastasis. Any of the anti-CD73 antibodies (e.g., 1E9 antibodies) described in WO 2017/100670, which is incorporated by reference in its entirety and for all purposes, may be used for the methods and compositions provided herein. The following embodiments described below are applicable to the methods and compositions provided herein including embodiments thereof.

The antibodies as provided herein are capable of binding a CD73 protein, activate and redistribute B cells and include the CDRs (CDR L1, CDR L2, CDR L3, CDR H1, CDR H2, and CDR H3) or functional fragments thereof of the mouse monoclonal antibody 1E9 (also referred to herein as the 1E9 antibody) (Thomson LF et al. Tissue Antigens 2008, Volume 35, Issue 1: Production and characterization of monoclonal antibodies to the glycosyl phosphatidylinositol-anchored lymphocyte differentiation antigen ecto-5′-nucleotidase (CD73)). The antibodies described herein including embodiments thereof maybe used for any of the methods or compositions described herein and below.

The anti-CD73 antibody provided herein may include a humanized light chain variable region including an 1E9 antibody CDR L1, an 1E9 antibody CDR L2, and an 1E9 antibody CDR L3 and a humanized heavy chain variable region including an 1E9 antibody CDR H1, an 1E9 antibody CDR H2, and an 1E9 antibody CDR H3. In aspects, the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and the CDR H3 has a sequence of SEQ ID NO:6. In aspects, the humanized light chain variable region includes at least one binding framework region residue. In aspects, the humanized heavy chain variable region includes at least one binding framework region residue. A framework region residue involved in (or important for) epitope binding (e.g. CD73 binding) is referred to herein as a binding framework region residue. The binding framework region residues may reside in the framework region of a humanized light chain variable region (i.e. FR L1, FR L2, FR L3, FR L4) or they may reside in the framework of a humanized heavy chain variable region (i.e. FR H1, FR H2, FR H3, FR H4). A binding framework residue residing in the FR L3 region of a humanized light chain is referred to herein as a FR L3 binding framework region residue. Thus, a binding framework region residue residing in the FR H3 region of a humanized heavy chain is referred to herein as a FR H3 binding framework region residue.

The anti-CD73 antibody provided herein may include a humanized light chain variable region and a humanized heavy chain variable region. The humanized light chain variable region may include: (i) a CDR L1 as set forth in SEQ ID NO:1, a CDR L2 as set forth in SEQ ID NO:2, a CDR L3 as set forth in SEQ ID NO:3 and (ii) a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87.

The humanized heavy chain variable region may include: (i) a mouse CDR H1 as set forth in SEQ ID NO:4, a mouse CDR H2 as set forth in SEQ ID NO:5, and a mouse CDR H3 as set forth in SEQ ID NO:6 and (ii) an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized light chain variable region includes a binding framework region residue that is a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87.

In embodiments, the humanized light chain variable region includes a binding framework region residue that is a valine at a position corresponding to Kabat position 2. In aspects, the humanized light chain variable region includes a binding framework region residue that is a methionine at a position corresponding to Kabat position 4. In aspects, the humanized light chain variable region includes a binding framework region residue that is an aspartic acid or a leucine at a position corresponding to Kabat position 9. In aspects, the humanized light chain variable region includes a binding framework region residue that is a proline or a serine at a position corresponding to Kabat position 12. In aspects, the humanized light chain variable region includes a binding framework region residue that is a lysine or a proline at a position corresponding to Kabat position 18. In aspects, the humanized light chain variable region includes a binding framework region residue that is a alanine at a position corresponding to Kabat position 43. In aspects, the humanized light chain variable region includes a binding framework region residue that is a proline or a serine at a position corresponding to Kabat position 60.

In embodiments, the humanized light chain variable region includes a binding framework region residue that is a threonine at a position corresponding to Kabat position 74. In aspects, the humanized light chain variable region includes a binding framework region residue that is an asparagine or a serine at a position corresponding to Kabat position 76. In aspects, the humanized light chain variable region includes a binding framework region residue that is an asparagine or a serine at a position corresponding to Kabat position 77. In aspects, the humanized light chain variable region includes a binding framework region residue that is an isoleucine or a leucine at a position corresponding to Kabat position 78. In aspects, the humanized light chain variable region includes a binding framework region residue that is a serine or an alanine at a position corresponding to Kabat position 80. In aspects, the humanized light chain variable region includes a binding framework region residue that is a glutamine at a position corresponding to Kabat position 100. In aspects, the humanized light chain variable region includes a binding framework region residue that is a valine at a position corresponding to Kabat position 104. In aspects, the humanized light chain variable region includes a binding framework region residue that is a glutamic acid or an alanine at a position corresponding to Kabat position 1. In aspects, the humanized light chain variable region includes a binding framework region residue that is a glutamine at a position corresponding to Kabat position 3.

In embodiments, the humanized light chain variable region includes a binding framework region residue that is a phenylalanine or a threonine at a position corresponding to Kabat position 10. In aspects, the humanized light chain variable region includes a binding framework region residue that is a glutamine at a position corresponding to Kabat position 11. In aspects, the humanized light chain variable region includes a binding framework region residue that is an alanine or a leucine at a position corresponding to Kabat position 13. In aspects, the humanized light chain variable region includes a binding framework region residue that is a threonine at a position corresponding to Kabat position 14. In aspects, the humanized light chain variable region includes a binding framework region residue that is a valine or a proline at a position corresponding to Kabat position 15. In aspects, the humanized light chain variable region includes a binding framework region residue that is a lysine at a position corresponding to Kabat position 16. In aspects, the humanized light chain variable region includes a binding framework region residue that is a glutamic acid or an aspartic acid at a position corresponding to Kabat position 17. In aspects, the humanized light chain variable region includes a binding framework region residue that is a threonine at a position corresponding to Kabat position 22.

In embodiments, the humanized light chain variable region includes a binding framework region residue that is a lysine at a position corresponding to Kabat position 42. In aspects, the humanized light chain variable region includes a binding framework region residue that is an arginine at a position corresponding to Kabat position 45. In aspects, the humanized light chain variable region includes a binding framework region residue that is an isoleucine at a position corresponding to Kabat position 58. In aspects, the humanized light chain variable region includes a binding framework region residue that is a tyrosine at a position corresponding to Kabat position 67. In aspects, the humanized light chain variable region includes a binding framework region residue that is a phenylalanine at a position corresponding to Kabat position 73. In aspects, the humanized light chain variable region includes a binding framework region residue that is an isoleucine at a position corresponding to Kabat position 78. In aspects, the humanized light chain variable region includes a binding framework region residue that is a tyrosine at a position corresponding to Kabat position 85. In aspects, the humanized light chain variable region includes a binding framework region residue that is a phenylalanine at a position corresponding to Kabat position 87.

The humanized heavy chain variable region provided herein may include a binding framework region residue that is an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized heavy chain variable region includes a binding framework region residue that is an isoleucine at a position corresponding to Kabat position 37. In aspects, the humanized heavy chain variable region includes a binding framework region residue that is an alanine or a proline at a position corresponding to Kabat position 40. In aspects, the humanized heavy chain variable region includes a binding framework region residue that is a lysine at a position corresponding to Kabat position 43. In aspects, the humanized heavy chain variable region includes a binding framework region residue that is a serine at a position corresponding to Kabat position 70. In aspects, the humanized heavy chain variable region includes a binding framework region residue that is an isoleucine or a threonine at a position corresponding to Kabat position 75. In aspects, the humanized heavy chain variable region includes a binding framework region residue that is a tryptophan at a position corresponding to Kabat position 82. In aspects, the humanized heavy chain variable region includes a binding framework region residue that is an arginine or a lysine at a position corresponding to Kabat position 83. In aspects, the humanized heavy chain variable region includes a binding framework region residue that is a alanine at a position corresponding to Kabat position 84.

In embodiments, the humanized heavy chain variable region includes a binding framework region residue that is a serine at a position corresponding to Kabat position 85. In aspects, the humanized heavy chain variable region includes a binding framework region residue that is a valine or a methionine at a position corresponding to Kabat position 89. In aspects, the humanized heavy chain variable region includes a binding framework region residue that is a valine at a position corresponding to Kabat position 5. In aspects, the humanized heavy chain variable region includes a binding framework region residue that is a serine at a position corresponding to Kabat position 7. In aspects, the humanized heavy chain variable region includes a binding framework region residue that is a valine at a position corresponding to Kabat position 11. In aspects, the humanized heavy chain variable region includes a binding framework region residue that is a glutamic acid or a lysine at a position corresponding to Kabat position 12. In aspects, the humanized heavy chain variable region includes a binding framework region residue that is an isoleucine or a valine at a position corresponding to Kabat position 20. In aspects, the humanized heavy chain variable region includes a binding framework region residue that is an arginine at a position corresponding to Kabat position 38. In aspects, the humanized heavy chain variable region includes a binding framework region residue that is an arginine at a position corresponding to Kabat position 66. In aspects, the humanized heavy chain variable region includes a binding framework region residue that is an valine at a position corresponding to Kabat position 67.

In embodiments, the humanized heavy chain variable region includes a binding framework region residue that is an isoleucine at a position corresponding to Kabat position 69. In aspects, the humanized heavy chain variable region includes a binding framework region residue that is an alanine at a position corresponding to Kabat position 71. In aspects, the humanized heavy chain variable region includes a binding framework region residue that is a lysine at a position corresponding to Kabat position 73. In aspects, the humanized heavy chain variable region includes a binding framework region residue that is a threonine at a position corresponding to Kabat position 87. In aspects, the humanized heavy chain variable region includes a binding framework region residue that is a glutamic acid at a position corresponding to Kabat position 1. In aspects, the humanized heavy chain variable region includes a binding framework region residue that is a valine at a position corresponding to Kabat position 24. In aspects, the humanized heavy chain variable region includes a binding framework region residue that is a arginine at a position corresponding to Kabat position 44. In aspects, the humanized heavy chain variable region includes a binding framework region residue that is a methionine at a position corresponding to Kabat position 48. In aspects, the humanized heavy chain variable region includes a binding framework region residue that is a leucine at a position corresponding to Kabat position 80. In aspects, the humanized heavy chain variable region includes a binding framework region residue that is a glutamic acid at a position corresponding to Kabat position 81.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, and a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, and a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, and a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, and a methionine at a position corresponding to Kabat position 89.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

In embodiments, the humanized heavy chain variable region includes the sequence of SEQ ID NO:7. In aspects, the humanized heavy chain variable region is SEQ ID NO:7. In aspects, the humanized light chain variable region includes the sequence of SEQ ID NO:8. In aspects, the humanized light chain variable region is SEQ ID NO:8. In aspects, the humanized heavy chain variable region includes the sequence of SEQ ID NO:7, and the humanized light chain variable region includes the sequence of SEQ ID NO:8. In aspects, the humanized heavy chain variable region is SEQ ID NO:7, and the humanized light chain variable region is SEQ ID NO:8.

The anti-CD73 antibodies as provided herein may be Fab′ fragments. Where the anti-CD73 antibodies are Fab′ fragments, the anti-CD73 antibodies include a humanized heavy chain (e.g. including a constant and a variable region) and a humanized light chain (e.g. including a constant and a variable region). In aspects, the anti-CD73 antibody is a Fab′ fragment. In aspects, the anti-CD73 antibody includes a human constant region. In aspects, the anti-CD73 antibody is an IgG. In aspects, the anti-CD73 antibody is an IgG1. In aspects, the anti-CD73 antibody is an IgG4. In aspects, the anti-CD73 antibody is an IgA. In aspects, the anti-CD73 antibody is an IgM.

In embodiments, the anti-CD73 antibody is a single chain antibody. A single chain antibody includes a variable light chain and a variable heavy chain. A person of skill in the art will immediately recognize that a single chain antibody includes a single light chain and a single heavy chain, in contrast to an immunoglobulin antibody, which includes two identical pairs of polypeptide chains, each pair having one light chain and one heavy chain. Each light chain and heavy chain in turn consists of two regions: a variable (“V”) region (i.e. variable light chain and variable heavy chain) involved in binding the target antigen, and a constant (“C”) region that interacts with other components of the immune system. The variable light chain and the variable heavy chain in a single chain antibody may be linked through a linker peptide. Examples for linker peptides of single chain antibodies are described in Bird, R. E., Hardman, K. D., Jacobson, J. W., Johnson, S., Kaufman, B. M., Lee, S. M., Lee, T., Pope, S. H., Riordan, G. S. and Whitlow, M. (1988). Methods of making scFv antibodies have been described. See, Huse et al., Science 246:1275-1281 (1989); Ward et al., Nature 341:544-546 (1989); and Vaughan et al., Nature Biotech. 14:309-314 (1996). Briefly, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell.

The ability of an antibody to bind a specific epitope (e.g., CD73) can be described by the equilibrium dissociation constant (K_{D}). The equilibrium dissociation constant (K_{D}) as defined herein is the ratio of the dissociation rate (K-off) and the association rate (K-on) of an anti-CD73 antibody to a CD73 protein. It is described by the following formula: K_{D}=K-off/K-on. In aspects, the anti-CD73 antibody is capable of binding a CD73 antigen with an equilibrium dissociation constant (K_{D}) from about 0.5 to about 25 nM. In aspects, the anti-CD73 antibody is capable of binding a CD73 antigen with an equilibrium dissociation constant (K_{D}) from about 1 to about 25 nM. In aspects, the anti-CD73 antibody is capable of binding a CD73 antigen with an equilibrium dissociation constant (K_{D}) from about 1.5 to about 25 nM. In aspects, the anti-CD73 antibody is capable of binding a CD73 antigen with an equilibrium dissociation constant (K_{D}) from about 2 to about 25 nM. In aspects, the anti-CD73 antibody is capable of binding a CD73 antigen with an equilibrium dissociation constant (K_{D}) from about 2.5 to about 25 nM. In aspects, the anti-CD73 antibody is capable of binding a CD73 antigen with an equilibrium dissociation constant (K_{D}) from about 3 to about 25 nM. In aspects, the anti-CD73 antibody is capable of binding a CD73 antigen with an equilibrium dissociation constant (K_{D}) from about 3.5 to about 25 nM. In aspects, the anti-CD73 antibody is capable of binding a CD73 antigen with an equilibrium dissociation constant (K_{D}) from about 4 to about 25 nM. In aspects, the anti-CD73 antibody is capable of binding a CD73 antigen with an equilibrium dissociation constant (K_{D}) in this paragraph at a pH below 7.5. In aspects, the anti-CD73 antibody is capable of binding a CD73 antigen with an equilibrium dissociation constant (K_{D}) in this paragraph at a pH of less than about 7.5. In aspects, the anti-CD73 antibody is capable of binding a CD73 antigen with an equilibrium dissociation constant (K_{D}) in this paragraph at a pH of less than about 7.0. In aspects, the anti-CD73 antibody is capable of binding a CD73 antigen with an equilibrium dissociation constant (K_{D}) in this paragraph at a pH of less than about 6.5. In aspects, the anti-CD73 antibody is capable of binding a CD73 antigen with an equilibrium dissociation constant (K_{D}) in this paragraph at a pH of less than about 6.0. In aspects, the anti-CD73 antibody is capable of binding a CD73 antigen with an equilibrium dissociation constant (K_{D}) in this paragraph at a pH of less than about 5.5. In aspects, the anti-CD73 antibody is capable of binding a CD73 antigen with an equilibrium dissociation constant (K_{D}) in this paragraph at a pH of less than about 5. In aspects, the anti-CD73 antibody is capable of binding a CD73 antigen with an equilibrium dissociation constant (K_{D}) in this paragraph at a pH of less than about 4.5. In aspects, the anti-CD73 antibody is capable of binding a CD73 antigen with an equilibrium dissociation constant (K_{D}) in this paragraph at a pH from about 6.0 to about 7.0. In aspects, the anti-CD73 antibody is capable of binding a CD73 antigen with an equilibrium dissociation constant (K_{D}) in this paragraph at a pH of about 6.0. In aspects, the anti-CD73 antibody is capable of binding a CD73 antigen with an equilibrium dissociation constant (K_{D}) in this paragraph at a pH of about 6.1. In aspects, the anti-CD73 antibody is capable of binding a CD73 antigen with an equilibrium dissociation constant (K_{D}) in this paragraph at a pH of about 6.2. In aspects, the anti-CD73 antibody is capable of binding a CD73 antigen with an equilibrium dissociation constant (K_{D}) in this paragraph at a pH of about 6.3. In aspects, the anti-CD73 antibody is capable of binding a CD73 antigen with an equilibrium dissociation constant (K_{D}) in this paragraph at a pH of about 6.4. In aspects, the anti-CD73 antibody is capable of binding a CD73 antigen with an equilibrium dissociation constant (K_{D}) in this paragraph at a pH of about 6.5. In aspects, the anti-CD73 antibody is capable of binding a CD73 antigen with an equilibrium dissociation constant (K_{D}) in this paragraph at a pH of about 6.6. In aspects, the anti-CD73 antibody is capable of binding a CD73 antigen with an equilibrium dissociation constant (K_{D}) in this paragraph at a pH of about 6.7. In aspects, the anti-CD73 antibody is capable of binding a CD73 antigen with an equilibrium dissociation constant (K_{D}) in this paragraph at a pH of about 6.8. In aspects, the anti-CD73 antibody is capable of binding a CD73 antigen with an equilibrium dissociation constant (K_{D}) in this paragraph at a pH of about 6.9. In aspects, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) from about 4.5 to about 25 nM. In aspects, the anti-CD73 antibody is capable of binding a CD73 antigen with an equilibrium dissociation constant (K_{D}) from about 5 to about 25 nM. In aspects, the anti-CD73 antibody is capable of binding a CD73 antigen with an equilibrium dissociation constant (K_{D}) from about 5.5 to about 25 nM. In aspects, the anti-CD73 antibody is capable of binding a CD73 antigen with an equilibrium dissociation constant (K_{D}) from about 6 to about 25 nM. In aspects, the anti-CD73 antibody is capable of binding a CD73 antigen with an equilibrium dissociation constant (K_{D}) from about 6.5 to about 25 nM. In aspects, the anti-CD73 antibody is capable of binding a CD73 antigen with an equilibrium dissociation constant (K_{D}) from about 7 to about 25 nM. In aspects, the anti-CD73 antibody is capable of binding a CD73 antigen with an equilibrium dissociation constant (K_{D}) from about 7.5 to about 25 nM. In aspects, the anti-CD73 antibody is capable of binding a CD73 antigen with an equilibrium dissociation constant (K_{D}) from about 8 to about 25 nM. In aspects, the anti-CD73 antibody is capable of binding a CD73 antigen with an equilibrium dissociation constant (K_{D}) in this paragraph at a pH below 7.5. In aspects, the anti-CD73 antibody is capable of binding a CD73 antigen with an equilibrium dissociation constant (K_{D}) in this paragraph at a pH of less than about 7.5. In aspects, the anti-CD73 antibody is capable of binding a CD73 antigen with an equilibrium dissociation constant (K_{D}) in this paragraph at a pH of less than about 7.0. In aspects, the anti-CD73 antibody is capable of binding a CD73 antigen with an equilibrium dissociation constant (K_{D}) in this paragraph at a pH of less than about 6.5. In aspects, the anti-CD73 antibody is capable of binding a CD73 antigen with an equilibrium dissociation constant (K_{D}) in this paragraph at a pH of less than about 6.0. In aspects, the anti-CD73 antibody is capable of binding a CD73 antigen with an equilibrium dissociation constant (K_{D}) in this paragraph at a pH of less than about 5.5. In aspects, the anti-CD73 antibody is capable of binding a CD73 antigen with an equilibrium dissociation constant (K_{D}) in this paragraph at a pH of less than about 5. In aspects, the anti-CD73 antibody is capable of binding a CD73 antigen with an equilibrium dissociation constant (K_{D}) in this paragraph at a pH of less than about 4.5. In aspects, the anti-CD73 antibody is capable of binding a CD73 antigen with an equilibrium dissociation constant (K_{D}) in this paragraph at a pH from about 6.0 to about 7.0. In aspects, the anti-CD73 antibody is capable of binding a CD73 antigen with an equilibrium dissociation constant (K_{D}) in this paragraph at a pH of about 6.0. In aspects, the anti-CD73 antibody is capable of binding a CD73 antigen with an equilibrium dissociation constant (K_{D}) in this paragraph at a pH of about 6.1. In aspects, the anti-CD73 antibody is capable of binding a CD73 antigen with an equilibrium dissociation constant (K_{D}) in this paragraph at a pH of about 6.2. In aspects, the anti-CD73 antibody is capable of binding a CD73 antigen with an equilibrium dissociation constant (K_{D}) in this paragraph at a pH of about 6.3. In aspects, the anti-CD73 antibody is capable of binding a CD73 antigen with an equilibrium dissociation constant (K_{D}) in this paragraph at a pH of about 6.4. In aspects, the anti-CD73 antibody is capable of binding a CD73 antigen with an equilibrium dissociation constant (K_{D}) in this paragraph at a pH of about 6.5. In aspects, the anti-CD73 antibody is capable of binding a CD73 antigen with an equilibrium dissociation constant (K_{D}) in this paragraph at a pH of about 6.6. In aspects, the anti-CD73 antibody is capable of binding a CD73 antigen with an equilibrium dissociation constant (K_{D}) in this paragraph at a pH of about 6.7. In aspects, the anti-CD73 antibody is capable of binding a CD73 antigen with an equilibrium dissociation constant (K_{D}) in this paragraph at a pH of about 6.8. In aspects, the anti-CD73 antibody is capable of binding a CD73 antigen with an equilibrium dissociation constant (K_{D}) in this paragraph at a pH of about 6.9. In aspects, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) from about 8.5 to about 25 nM. In aspects, the anti-CD73 antibody is capable of binding a CD73 antigen with an equilibrium dissociation constant (K_{D}) from about 9 to about 25 nM. In aspects, the anti-CD73 antibody is capable of binding a CD73 antigen with an equilibrium dissociation constant (K_{D}) from about 9.5 to about 25 nM. In aspects, the anti-CD73 antibody is capable of binding a CD73 antigen with an equilibrium dissociation constant (K_{D}) from about 10 to about 25 nM. In aspects, the anti-CD73 antibody is capable of binding a CD73 antigen with an equilibrium dissociation constant (K_{D}) from about 11 to about 25 nM. In aspects, the anti-CD73 antibody is capable of binding a CD73 antigen with an equilibrium dissociation constant (K_{D}) from about 12 to about 25 nM. In aspects, the anti-CD73 antibody is capable of binding a CD73 antigen with an equilibrium dissociation constant (K_{D}) from about 13 to about 25 nM. In aspects, the anti-CD73 antibody is capable of binding a CD73 antigen with an equilibrium dissociation constant (K_{D}) from about 14 to about 25 nM. In aspects, the anti-CD73 antibody is capable of binding a CD73 antigen with an equilibrium dissociation constant (K_{D}) from about 15 to about 25 nM. In aspects, the anti-CD73 antibody is capable of binding a CD73 antigen with an equilibrium dissociation constant (K_{D}) from about 16 to about 25 nM. In aspects, the anti-CD73 antibody is capable of binding a CD73 antigen with an equilibrium dissociation constant (K_{D}) in this paragraph at a pH below 7.5. In aspects, the anti-CD73 antibody is capable of binding a CD73 antigen with an equilibrium dissociation constant (K_{D}) in this paragraph at a pH of less than about 7.5. In aspects, the anti-CD73 antibody is capable of binding a CD73 antigen with an equilibrium dissociation constant (K_{D}) in this paragraph at a pH of less than about 7.0. In aspects, the anti-CD73 antibody is capable of binding a CD73 antigen with an equilibrium dissociation constant (K_{D}) in this paragraph at a pH of less than about 6.5. In aspects, the anti-CD73 antibody is capable of binding a CD73 antigen with an equilibrium dissociation constant (K_{D}) in this paragraph at a pH of less than about 6.0. In aspects, the anti-CD73 antibody is capable of binding a CD73 antigen with an equilibrium dissociation constant (K_{D}) in this paragraph at a pH of less than about 5.5. In aspects, the anti-CD73 antibody is capable of binding a CD73 antigen with an equilibrium dissociation constant (K_{D}) in this paragraph at a pH of less than about 5. In aspects, the anti-CD73 antibody is capable of binding a CD73 antigen with an equilibrium dissociation constant (K_{D}) in this paragraph at a pH of less than about 4.5. In aspects, the anti-CD73 antibody is capable of binding a CD73 antigen with an equilibrium dissociation constant (K_{D}) in this paragraph at a pH from about 6.0 to about 7.0. In aspects, the anti-CD73 antibody is capable of binding a CD73 antigen with an equilibrium dissociation constant (K_{D}) in this paragraph at a pH of about 6.0. In aspects, the anti-CD73 antibody is capable of binding a CD73 antigen with an equilibrium dissociation constant (K_{D}) in this paragraph at a pH of about 6.1. In aspects, the anti-CD73 antibody is capable of binding a CD73 antigen with an equilibrium dissociation constant (K_{D}) in this paragraph at a pH of about 6.2. In aspects, the anti-CD73 antibody is capable of binding a CD73 antigen with an equilibrium dissociation constant (K_{D}) in this paragraph at a pH of about 6.3. In aspects, the anti-CD73 antibody is capable of binding a CD73 antigen with an equilibrium dissociation constant (K_{D}) in this paragraph at a pH of about 6.4. In aspects, the anti-CD73 antibody is capable of binding a CD73 antigen with an equilibrium dissociation constant (K_{D}) in this paragraph at a pH of about 6.5. In aspects, the anti-CD73 antibody is capable of binding a CD73 antigen with an equilibrium dissociation constant (K_{D}) in this paragraph at a pH of about 6.6. In aspects, the anti-CD73 antibody is capable of binding a CD73 antigen with an equilibrium dissociation constant (K_{D}) in this paragraph at a pH of about 6.7. In aspects, the anti-CD73 antibody is capable of binding a CD73 antigen with an equilibrium dissociation constant (K_{D}) in this paragraph at a pH of about 6.8. In aspects, the anti-CD73 antibody is capable of binding a CD73 antigen with an equilibrium dissociation constant (K_{D}) in this paragraph at a pH of about 6.9. In aspects, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) from about 17 to about 25 nM. In aspects, the anti-CD73 antibody is capable of binding a CD73 antigen with an equilibrium dissociation constant (K_{D}) from about 18 to about 25 nM. In aspects, the anti-CD73 antibody is capable of binding a CD73 antigen with an equilibrium dissociation constant (K_{D}) from about 19 to about 25 nM. In aspects, the anti-CD73 antibody is capable of binding a CD73 antigen with an equilibrium dissociation constant (K_{D}) from about 20 to about 25 nM. In aspects, the anti-CD73 antibody is capable of binding a CD73 antigen with an equilibrium dissociation constant (K_{D}) from about 21 to about 25 nM. In aspects, the anti-CD73 antibody is capable of binding a CD73 antigen with an equilibrium dissociation constant (K_{D}) from about 22 to about 25 nM. In aspects, the anti-CD73 antibody is capable of binding a CD73 antigen with an equilibrium dissociation constant (K_{D}) from about 23 to about 25 nM. In aspects, the anti-CD73 antibody is capable of binding a CD73 antigen with an equilibrium dissociation constant (K_{D}) from about 24 to about 25 nM. In aspects, the anti-CD73 antibody is capable of binding a CD73 antigen with an equilibrium dissociation constant (K_{D}) of about 0.5, 1 2, 3, 4, 5, 6, 7, 8, 9, 10, 11, 12, 13, 14, 15, 16, 17, 18, 19, 20, 21, 22, 23, 24, or 25 nM. In aspects, the anti-CD73 antibody is capable of binding a CD73 antigen with an equilibrium dissociation constant (K_{D}) in this paragraph at a pH below 7.5. In aspects, the anti-CD73 antibody is capable of binding a CD73 antigen with an equilibrium dissociation constant (K_{D}) in this paragraph at a pH of less than about 7.5. In aspects, the anti-CD73 antibody is capable of binding a CD73 antigen with an equilibrium dissociation constant (K_{D}) in this paragraph at a pH of less than about 7.0. In aspects, the anti-CD73 antibody is capable of binding a CD73 antigen with an equilibrium dissociation constant (K_{D}) in this paragraph at a pH of less than about 6.5. In aspects, the anti-CD73 antibody is capable of binding a CD73 antigen with an equilibrium dissociation constant (K_{D}) in this paragraph at a pH of less than about 6.0. In aspects, the anti-CD73 antibody is capable of binding a CD73 antigen with an equilibrium dissociation constant (K_{D}) in this paragraph at a pH of less than about 5.5. In aspects, the anti-CD73 antibody is capable of binding a CD73 antigen with an equilibrium dissociation constant (K_{D}) in this paragraph at a pH of less than about 5. In aspects, the anti-CD73 antibody is capable of binding a CD73 antigen with an equilibrium dissociation constant (K_{D}) in this paragraph at a pH of less than about 4.5. In aspects, the anti-CD73 antibody is capable of binding a CD73 antigen with an equilibrium dissociation constant (K_{D}) in this paragraph at a pH from about 6.0 to about 7.0. In aspects, the anti-CD73 antibody is capable of binding a CD73 antigen with an equilibrium dissociation constant (K_{D}) in this paragraph at a pH of about 6.0. In aspects, the anti-CD73 antibody is capable of binding a CD73 antigen with an equilibrium dissociation constant (K_{D}) in this paragraph at a pH of about 6.1. In aspects, the anti-CD73 antibody is capable of binding a CD73 antigen with an equilibrium dissociation constant (K_{D}) in this paragraph at a pH of about 6.2. In aspects, the anti-CD73 antibody is capable of binding a CD73 antigen with an equilibrium dissociation constant (K_{D}) in this paragraph at a pH of about 6.3. In aspects, the anti-CD73 antibody is capable of binding a CD73 antigen with an equilibrium dissociation constant (K_{D}) in this paragraph at a pH of about 6.4. In aspects, the anti-CD73 antibody is capable of binding a CD73 antigen with an equilibrium dissociation constant (K_{D}) in this paragraph at a pH of about 6.5. In aspects, the anti-CD73 antibody is capable of binding a CD73 antigen with an equilibrium dissociation constant (K_{D}) in this paragraph at a pH of about 6.6. In aspects, the anti-CD73 antibody is capable of binding a CD73 antigen with an equilibrium dissociation constant (K_{D}) in this paragraph at a pH of about 6.7. In aspects, the anti-CD73 antibody is capable of binding a CD73 antigen with an equilibrium dissociation constant (K_{D}) in this paragraph at a pH of about 6.8. In aspects, the anti-CD73 antibody is capable of binding a CD73 antigen with an equilibrium dissociation constant (K_{D}) in this paragraph at a pH of about 6.9. In aspects, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) of about 7.1 nM. In aspects, the anti-CD73 antibody is capable of binding a CD73 antigen with an equilibrium dissociation constant (K_{D}) of about 6.9 nM. In aspects, the anti-CD73 antibody is capable of binding a CD73 antigen with an equilibrium dissociation constant (K_{D}) of about 9.4 nM. In aspects, the anti-CD73 antibody is capable of binding a CD73 antigen with an equilibrium dissociation constant (K_{D}) of about 19.5 nM. In aspects, the anti-CD73 antibody is capable of binding a CD73 antigen with an equilibrium dissociation constant (K_{D}) of about 17.8 nM. In aspects, the anti-CD73 antibody is capable of binding a CD73 antigen with an equilibrium dissociation constant (K_{D}) of about 15.9 nM. In aspects, the anti-CD73 antibody is capable of binding a CD73 antigen with an equilibrium dissociation constant (K_{D}) in this paragraph at a pH below 7.5. In aspects, the anti-CD73 antibody is capable of binding a CD73 antigen with an equilibrium dissociation constant (K_{D}) in this paragraph at a pH of less than about 7.5. In aspects, the anti-CD73 antibody is capable of binding a CD73 antigen with an equilibrium dissociation constant (K_{D}) in this paragraph at a pH of less than about 7.0. In aspects, the anti-CD73 antibody is capable of binding a CD73 antigen with an equilibrium dissociation constant (K_{D}) in this paragraph at a pH of less than about 6.5. In aspects, the anti-CD73 antibody is capable of binding a CD73 antigen with an equilibrium dissociation constant (K_{D}) in this paragraph at a pH of less than about 6.0. In aspects, the anti-CD73 antibody is capable of binding a CD73 antigen with an equilibrium dissociation constant (K_{D}) in this paragraph at a pH of less than about 5.5. In aspects, the anti-CD73 antibody is capable of binding a CD73 antigen with an equilibrium dissociation constant (K_{D}) in this paragraph at a pH of less than about 5. In aspects, the anti-CD73 antibody is capable of binding a CD73 antigen with an equilibrium dissociation constant (K_{D}) in this paragraph at a pH of less than about 4.5. In aspects, the anti-CD73 antibody is capable of binding a CD73 antigen with an equilibrium dissociation constant (K_{D}) in this paragraph at a pH from about 6.0 to about 7.0. In aspects, the anti-CD73 antibody is capable of binding a CD73 antigen with an equilibrium dissociation constant (K_{D}) in this paragraph at a pH of about 6.0. In aspects, the anti-CD73 antibody is capable of binding a CD73 antigen with an equilibrium dissociation constant (K_{D}) in this paragraph at a pH of about 6.1. In aspects, the anti-CD73 antibody is capable of binding a CD73 antigen with an equilibrium dissociation constant (K_{D}) in this paragraph at a pH of about 6.2. In aspects, the anti-CD73 antibody is capable of binding a CD73 antigen with an equilibrium dissociation constant (K_{D}) in this paragraph at a pH of about 6.3. In aspects, the anti-CD73 antibody is capable of binding a CD73 antigen with an equilibrium dissociation constant (K_{D}) in this paragraph at a pH of about 6.4. In aspects, the anti-CD73 antibody is capable of binding a CD73 antigen with an equilibrium dissociation constant (K_{D}) in this paragraph at a pH of about 6.5. In aspects, the anti-CD73 antibody is capable of binding a CD73 antigen with an equilibrium dissociation constant (K_{D}) in this paragraph at a pH of about 6.6. In aspects, the anti-CD73 antibody is capable of binding a CD73 antigen with an equilibrium dissociation constant (K_{D}) in this paragraph at a pH of about 6.7. In aspects, the anti-CD73 antibody is capable of binding a CD73 antigen with an equilibrium dissociation constant (K_{D}) in this paragraph at a pH of about 6.8. In aspects, the anti-CD73 antibody is capable of binding a CD73 antigen with an equilibrium dissociation constant (K_{D}) in this paragraph at a pH of about 6.9. In aspects, the anti-CD73 antibody is capable of binding a CD73 antigen with an equilibrium dissociation constant (K_{D}) in this paragraph at a pH of about 7.0.

The anti-CD73 antibody provided herein including embodiments thereof may include a glutamine at a position corresponding to Kabat position 297. In aspects, the anti-CD73 antibody is bound to a CD73 antigen. In aspects, the CD73 antigen forms part of a cell. In aspects, the cell is a lymphoid cell. In aspects, the cell is a B cell.

Methods of Immunostimulation and B cell Modulation

The anti-CD73 antibodies used for the methods and included in the compositions provided herein including embodiments thereof are, inter alia, capable of binding CD73 proteins and inhibiting CD73 catalytic activity thereby preventing metastasis. Provided herein are novel methods and compositions based on Applicants' surprising finding that the anti-CD73 antibodies described herein not only revert immunosuppression of T cells, but have a surprising and previously unknown direct effect on B cells. Applicants are the first to describe that 1E9 anti-CD73 antibodies, in contrast to other known anti-CD73 antibodies (e.g., MEDI9447, AD2), are capable of activating B cells, and affecting the redistribution of B cells from the periphery to lymphoid tissues/organs. Applicants show that direct B cell activation occurs specifically with Applicants' anti-CD73 antibody, while other anti-CD73 do not show this effect. This previously unknown and unique feature of anti-CD73 antibodies may be useful for the treatment of various indications, for example, enhancing immunity to immunogenic cancers, treating autoimmune disease (e.g., multiple sclerosis), inflammatory diseases, or infectious disease.

Any of the embodiments described above for anti-CD73 antibodies may be applicable to the methods provided herein including embodiments thereof. Therefore, the anti-CD73 antibody used for the compositions provided herein may, for example, include heavy and light CDRs, wherein the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and said CDR H3 has a sequence of SEQ ID NO:6; include a humanized heavy chain variable region including the sequence of SEQ ID NO:7; is an IgG4; binds a CD73 antigen with an equilibrium dissociation constant (K_{D}) from about 0.3 to about 25 nM; optionally further include a glutamine at a position corresponding to Kabat position 297, or optionally forms part of a cell, wherein the cell is a B cell. In aspects, the anti-CD73 antibody used in the methods described herein is CPI-006.

In an aspect, a method of immunostimulating a subject is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006. In aspects, the anti-CD73 antibody is CPI-006. The term “immunostimulating” or “immunostimulation” as provided herein refers to the ability to activate the immune system or increasing activity of any of its components. In aspects, the method of immunostimulating a subject comprises increasing inflammatory cytokines in the subject. In aspects, the method of immunostimulating a subject comprises increasing inflammatory cytokines in the subject, wherein the inflammatory cytokines are TNF-α, TNF-β, MIP-1α, MIP-1β, IL-6, IL-10, IL-8, IP-10, MCP-1, MCP-2, IL-1Ra, GRO-α, MIP-3α, TNF-RII, IL-7, MMP-9, CRP, SAA, MMP-3, MDC, YKL-40, IL-27, or a combination of two or more thereof. In aspects, the method of immunostimulating a subject comprises increasing inflammatory cytokines in the subject, wherein the inflammatory cytokines are TNF-α, TNF-β, MIP-1α, MIP-1β, IL-6, IL-10, IL-8, IP-10, MCP-1, MCP-2, IL-1Ra, GRO-α, MIP-3α, TNF-RII, IL-7, MMP-9, or a combination of two or more thereof. In aspects, the method of immunostimulating a subject comprises increasing inflammatory cytokines in the subject, wherein the inflammatory cytokines are TNF-α, TNF-β,MIP-1α, MIP-1β, IL-6, IL-10, IL-8, IP-10, MCP-1, MCP-2, IL-1Ra, GRO-α, MIP-3α, or a combination of two or more thereof; and optionally wherein the inflammatory cytokines have a loge fold increase of at least two from about 0.5 hours to about 2 hours after administration of the anti-CD73 antibody. In aspects, the method of immunostimulating a subject comprises increasing inflammatory cytokines in the subject, wherein the inflammatory cytokines are CRP, SAA, MMP-3, MDC, YKL-40, IL-27, or a combination of two or more thereof. In aspects, the method of immunostimulating a subject comprises increasing inflammatory cytokines in the subject, wherein the inflammatory cytokines are C-reactive protein (CRP), serum amyloid A (SAA), or a combination thereof; and optionally wherein the inflammatory cytokines have a loge fold increase of at least two from about 1 day to about 8 days after administration of the anti-CD73 antibody. In aspects, the method of immunostimulating a subject comprises increasing activation markers in the subject. In aspects, the method of immunostimulating a subject comprises increasing antigen presenting cells in the subject. In aspects, the method of immunostimulating a subject comprises increasing B cells in the subject. In aspects, the method of immunostimulating a subject comprises increasing dendritic cells in the subject. In aspects, the method of immunostimulating a subject comprises increasing antigen presenting cells in the subject, wherein the antigen-presenting cells express (i.e., comprise) CD3, CD14, CD19, CD25, CD69, CD83, CD86, MHC Class II (e.g., HLA-DR), BDCA-2, BDCA-4, CD11c^{low}, CD45RA, CD123, ILT-7, TLR7, TLR9, or a combination of two or more thereof. In aspects, the method of immunostimulating a subject comprises decreasing monocytes in the subject, e.g., in the blood of the subject. In aspects, the method of immunostimulating a subject comprises decreasing CD73^{NEG} CD8 T cells in the subject, e.g., in the blood of the subject. In aspects, the method of immunostimulating a subject comprises decreasing CD73^{POS} CD8 T cells in the subject, e.g., in the blood of the subject. In aspects, the method of immunostimulating a subject comprises increasing CD73^{NEG} CD4 T cells in the subject, e.g., in the blood of the subject. In aspects, the method of immunostimulating a subject comprises decreasing CD73^{POS} CD4 T cells in the subject, e.g., in the blood of the subject. In aspects, the method of immunostimulating a subject comprises increasing the CD4/CD8 ratio in the subject, e.g., in the blood of the subject. In aspects, the method of immunostimulating a subject comprises increasing the CD73^{NEG} CD4/CD73^{NEG} CD8 ratio in the subject, e.g., in the blood of the subject.

In aspects, the method of immunostimulating a subject in need thereof further comprises obtaining a biological sample from the subject, and monitoring activation markers in the biological sample. In aspects, the activation markers are B cells, dendritic cells, CD69, CD83, CD25, or a combination of two or more thereof. In aspects, the activation marker is a B cell. In aspects, the activation marker is a dendritic cell. In aspects, the activation marker is CD69. In aspects, the activation marker is CD83. In aspects, the activation marker is CD25. In aspects, the activation marker in the biological sample is increased relative to a control (e.g., relative to the activation markers in a biological sample taken prior to administration of an anti-CD73 antibody). In aspects, the methods further comprise administering an effective amount of the anti-CD73 antibody when the activation marker is increased relative to a control.

In aspects, the method of immunostimulating a subject in need thereof further comprises obtaining a biological sample from the subject, and monitoring antigen-presenting cells in the biological sample. In aspects the antigen-presenting cells are B cells. In aspects the antigen-presenting cells are dendritic cells. In aspects, the antigen-presenting cells comprise CD3, CD14, CD19, CD25, CD69, CD83, CD86, MHC Class II (e.g., HLA-DR), BDCA-2, BDCA-4, CD11c^{low}, CD45RA, CD123, ILT-7, TLR7, TLR9, or a combination of two or more thereof. In aspects, the antigen-presenting cells comprise CD3, CD14, CD19, CD86, MHC Class II (e.g., HLA-DR), BDCA-2, BDCA-4, CD11c^{low}, CD45RA, CD123, ILT-7, TLR7, TLR9, or a combination of two or more thereof. In aspects, the antigen-presenting cells comprise CD3, CD14, CD86, MHC Class II (e.g., HLA-DR), BDCA-2, BDCA-4, CD11c^{low}, CD45RA, CD123, ILT-7, TLR7, TLR9, or a combination of two or more thereof. In aspects, the antigen-presenting cells comprise CD86. In aspects, the antigen-presenting cells comprise MHC Class II. In aspects, the antigen-presenting cells comprise HLA-DR. In aspects, the antigen-presenting cells in the biological sample are increased relative to a control (e.g., relative to the antigen-presenting cells in a biological sample taken prior to administration of an anti-CD73 antibody). In aspects, the methods further comprise administering an effective amount of the anti-CD73 antibody when the antigen-presenting cells are increased relative to a control.

In aspects, the method of immunostimulating a subject in need thereof further comprises obtaining a biological sample (e.g., blood sample) from the subject, and monitoring monocytes, CD73^{POS} CD4 T cells, CD73^{NEG} CD4 T cells, CD73^{POS} CD8 T cells, CD73^{NEG} CD8 T cells, or a combination of two or more thereof in the biological sample. In aspects, the monocytes, CD73^{POS} CD8 T cells, CD73^{NEG} CD8 T cells, or a combination of two or more thereof, in the biological sample are decreased relative to a control (e.g., relative to the monocytes CD73^{POS} CD8 T cells, CD73^{NEG} CD8 T cells, respectively, in a biological sample taken prior to administration of an anti-CD73 antibody). In aspects, the methods further comprise administering an effective amount of the anti-CD73 antibody when the monocytes, CD73^{POS} CD8 T cells, CD73^{NEG} CD8 T cells, or a combination of two or more thereof, are decreased relative to a control. In aspects, the CD73^{POS} CD4 T cells, CD73^{NEG} CD4 T cells, or both, in the biological sample are increased relative to a control (e.g., relative to the CD73^{POS} CD4 T cells or CD73^{NEG} CD4 T cells, respectively, in a biological sample taken prior to administration of an anti-CD73 antibody). In aspects, the methods further comprise administering an effective amount of the anti-CD73 antibody when the CD73^{POS} CD4 T cells, CD73^{NEG} CD4 T cells, or both, are decreased relative to a control.

In an aspect, a method of activating B cells in a subject is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006. The term “B-cell activation” or “activating B cells” as provided herein are terms well known in the art and are used according to their conventional meaning in the art. For example, B cell activation involves binding of B cell receptors (BCRs) to an antigen, or T cells providing an activation signal to the B cell (thymus-dependent activation). Upon activation, B cells upregulate the expression of specific B cell activation marker proteins (e.g., CD69, CD83), which are characteristic of activated B cells.

In an aspect, a method of redistributing peripheral B cells in a subject is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006. The term “redistributing” or “redistribution” as provided herein refers to an effect a compound (e.g., anti-CD73 antibody as provided herein) has on the location of B cells in an organism. In aspects, the compound (e.g., anti-CD73 antibody as provided herein) binds to CD73 expressing B cells and modulates the location of a B cell in an organism relative to a standard control. In aspects, where the compound (e.g., anti-CD73 antibody as provided herein) modulates the location of a B cell in an organism, there are less B cells in the periphery (e.g., peripheral blood) of an organism relative to the absence of the compound. In aspects, the amount of B cells (number of B cells) present in peripheral blood of the subject is less relative to the absence of the anti-CD73 antibody. In aspects, the amount (number) of B cells present in primary lymphoid organs (e.g., spleen or thymus) of the subject is greater relative to the absence of the anti-CD73 antibody.

In an aspect, a method of decreasing egress of B cells from lymphoid tissue in a subject relative to a standard control is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006. A “lymphoid tissue” as provided herein includes any diffuse or unencapsulated bundles of lymphatic cells. Lymphoid tissue consists of lymphocytes and macrophages associated with a reticular fiber network and includes, without limitation, lymphatic nodules, Peyer's patches and tonsils.

A “standard control” as provided herein refers to a sample that serves as a reference, usually a known reference, for comparison to a test sample. For example, a test sample can be taken from a patient suspected of having a disease (e.g., an autoimmune disease, an inflammatory disease, cancer) and compared to samples from a patient known to have the disease, or a known normal (non-disease) individual. A control can also represent an average value gathered from a population of similar individuals, e.g., disease patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

In some examples of the disclosed methods, when the amount (number) of B cells present in lymphoid tissues or lymphoid organs or the B cell activity is assessed or the egress of B cells from lymphoid tissues is determined, the amount (number) is compared with a control amount (number) of B cells or B cell activity (e.g., in a healthy subject or in an untreated subject). By control is meant the amount (number) of B cells in lymphoid tissues or lymphoid organs or B cell activity from a sample or subject lacking the disease, a sample or subject at a selected stage of the disease or disease state, or in the absence of a particular variable such as a therapeutic agent. Alternatively, the control includes a known amount (number) of B cells present in lymphoid tissues or organs or B cell activity. Such a known amount correlates with an average level of subjects lacking the disease, at a selected stage of the disease or disease state, or in the absence of a particular variable such as a therapeutic agent. A control also includes the amount (number) of B cells present in lymphoid tissues or lymphoid organs or B cell activity from one or more selected samples or subjects as described herein. For example, a control includes an assessment of the amount (number) of B cells or B cell activity in a sample from a subject that does not have the disease, is at a selected stage of disease or disease state, or has not received treatment for the disease. Another exemplary control level includes an assessment of the amount (number) of B cells or B cell activity in samples taken from multiple subjects that do not have the disease, are at a selected stage of the disease, or have not received treatment for the disease.

When the control amount (number) of B cells or B cell activity includes the amount of B cells or B cell activity in a sample or subject in the absence of a therapeutic agent, the control sample or subject is optionally the same sample or subject to be tested before or after treatment with a therapeutic agent or is a selected sample or subject in the absence of the therapeutic agent.

Alternatively, a standard control is an average expression level calculated from a number of subjects without a particular disease. A control level also includes a known control level or value known in the art.

In an aspect, a method of increasing retention of B cells in lymphoid organs in a subject relative to a standard control is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006. A “lymphoid organ” as provided herein is a term well known in the art and is used herein according to its conventional meaning in the art. Examples of lymphoid organs include the thymus and the spleen. In aspects, the compound (e.g., anti-CD73 antibody as provided herein) modulates the location of a B cell in an organism relative to a standard control. In aspects, where the compound (e.g., anti-CD73 antibody as provided herein) modulates the location of a B cell in an organism, there are more B cells in a lymphoid organ (e.g., spleen) of an organism relative to the absence of the compound. In aspects, the amount of B cells in primary lymphoid organs (e.g., spleen or thymus) of the subject is greater relative to the absence of the anti-CD73 antibody.

In an aspect, a method of increasing internalization of a Sphingosine-1-phosphate receptor 1 (S1PR1) in a cell in a subject relative to a standard control is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006.

In embodiments, the subject is a subject having a cancer. In aspects, the subject is immune deficient. In aspects, the subject has an immune deficiency disease or an autoimmune disease.

Methods of Treatment

The methods provided herein are, inter alia, useful for treating diseases associated with or related to S1PR1 function, infectious diseases, inflammatory diseases, and autoimmune diseases.

Any of the embodiments described above for anti-CD73 antibodies may be applicable to the methods provided herein including embodiments thereof. Therefore, the anti-CD73 antibody used for the compositions provided herein may, for example, include heavy and light CDRs, wherein the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and said CDR H3 has a sequence of SEQ ID NO:6; include a humanized heavy chain variable region including the sequence of SEQ ID NO:7; may be an IgG4; may be capable of binding a CD73 antigen with an equilibrium dissociation constant (K_{D}) from about 0.3 to about 25 nM; may further include a glutamine at a position corresponding to Kabat position 297 or may form part of a cell, wherein the cell is a B cell.

The terms “Sphingosine-1-phosphate receptor 1”, “S1P1” or “S1PR1” as provided to herein include any of the recombinant or naturally-occurring forms of the Sphingosine-1-phosphate receptor 1 (S1PR1), also known as endothelial differentiation gene 1 (EDG1) or variants or homologs thereof that maintain S1PR1 activity (e.g. within at least 50%, 80%, 90%, 95%, 96%, 97%, 98%, 99% or 100% activity compared to S1PR1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring S1PR1 protein. In aspects, the S1PR1 protein is substantially identical to the protein identified by the UniProt reference number P21453 or a variant or homolog having substantial identity thereto.

A “Sphingosine-1-phosphate receptor 1 (S1PR1)-associated disease” as provided herein is used to broadly refer to disorders or symptoms of disease associated with S1PR1 function. In aspects, the disease is caused by, or a symptom of the disease is caused by aberrant S1PR1 function. S1PR1 function as described herein refers to any cellular function affected by S1PR1 and includes without limitation regulating endothelial cell cytoskeletal structure, migration, capillary-like network formation, vascular maturation, regulation of lymphocyte maturation, lymphocyte migration and lymphocyte trafficking.

The term “internalization” as provided herein refers to the cellular uptake of a molecule (e.g., S1PR1) through endocytosis. Endocytosis as provided herein refers to the cellular process of a molecule being surrounded by an area of plasma membrane thereby forming an ingested molecule. The area of plasma membrane including the ingested molecule subsequently buds off inside the cell to form a vesicle containing the ingested molecule.

Thus, in an aspect, a method of treating a Sphingosine-1-phosphate receptor 1 (S1PR1)-associated disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the S1PR1-associated disease is a graft versus host disease, an autoimmune disease, an inflammatory disease, viral myocarditis, a viral disease caused by viral myocarditis, a demyelinating disease, or organ or tissue transplant rejection. In aspects, the S1PR1-associated disease is an autoimmune disease. In aspects, the S1PR1-associated disease is multiple sclerosis, rheumatoid arthritis, atopic eczema (atopic dermatitis), Behçet's disease, uvea diseases, systemic lupus erythematosus, Sjogren's syndrome, polysclerosis, myasthenia gravis, diabetes type I, endocrine eye disorders, primary biliary cirrhosis, Crohn's disease, glomerulonephritis, sarcoidosis, psoriasis, pemphigus, aplastic anemia, idiopathic thrombocytopenic purpura, allergy, polyarteritis nodosa, progressive systemic sclerosis, mixed connective-tissue disease, aortitis syndrome, polymyositis, dermatomyositis, Wegener's granulomatosis, ulcerative colitis, active chronic hepatitis, autoimmune hemolytic anemia, Evans syndrome, bronchial asthma, liver failure, renal failure, or pollinosis.

In an aspect, a method of treating an infectious disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006.

In an aspect, a method of treating an inflammatory disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006.

In an aspect, a method of treating an autoimmune disease in a subject in need thereof is provided. The method includes administering to the subject an effective amount of an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006.

Methods of Cancer Treatment

In embodiments, the disclosure provides methods of treating cancer in a patient in need thereof comprising administering to the patient an effective amount of an anti-CD73 antibody to effectively activate an antigen-presenting cell. In aspects, the disclosure provides methods of treating cancer in a patient in need thereof comprising: administering to the patient an effective amount of an anti-CD73 antibody; and monitoring a level of an antigen-presenting cell. In aspects, the disclosure provides method of treating cancer in a patient in need thereof comprising: administering to the patient an effective amount of an anti-CD73 antibody to effectively activate an antigen-presenting cell; and monitoring a level of the antigen-presenting cell. In aspects, the level of the antigen-presenting cell in the biological sample is monitored by flow cytometry, immunohistochemistry, or a combination thereof. In aspects, the antigen-presenting cell is in a cancer tumor microenvironment. In aspects, the level of the antigen-presenting cell is monitored through a biological sample from the patient, such as a blood sample, such as a peripheral blood sample. In aspects, the biological sample comprises B cells in an amount of about 0.2×10e6 cells/mL or more, as measured by flow cytometry. In aspects, the biological sample comprises B cells in an amount of about 0.3×10e6 cells/mL or more, as measured by flow cytometry. In aspects, the biological sample comprises B cells in an amount of about 0.4×10e6 cells/mL or more, as measured by flow cytometry. In aspects, the biological sample comprises B cells in an amount of about 0.5×10e6 cells/mL or more, as measured by flow cytometry. In aspects, the biological sample comprises B cells in an amount of about 0.6×10e6 cells/mL or more, as measured by flow cytometry. In aspects, the biological sample comprises dendritic cell in an amount of about 0.03×10e6 cell/mL or more, as measured by flow cytometry. In aspects, the biological sample comprises dendritic cell in an amount of about 0.04×10e6 cell/mL or more, as measured by flow cytometry. In aspects, the biological sample comprises dendritic cell in an amount of about 0.05×10e6 cell/mL or more, as measured by low cytometry. In aspects, the biological sample comprises dendritic cell in an amount of about 0.06×10e6 cell/mL or more, as measured by flow cytometry. In aspects, the biological sample comprises dendritic cell in an amount of about 0.07×10e6 cell/mL or more, as measured by flow cytometry. In aspects, the antigen presenting cell is a B cell. In aspects, the antigen presenting cell is a B cell that is CD19+. In aspects, the antigen presenting cell is a dendritic cell. In aspects, the antigen presenting cell is a classical dendritic cell. In aspects, the antigen presenting cell is a plasmacytoid dendritic cell. In aspects, the antigen presenting cell is positive for CD3, CD14, CD19, CD69, CD83, CD86, MHC Class II (e.g., HLA-DR), BDCA-2, BDCA-4, CD11c^{low}, CD45RA, CD123, ILT-7, TLR7, TLR9, or a combination of two or more thereof. In aspects, the anti-CD73 antibody is any described herein. In aspects, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006.

In embodiments, the disclosure provides methods of treating cancer in a patient in need thereof comprising administering to the patient an effective amount of an anti-CD73 antibody to effectively activate an antigen-presenting cell; obtaining a biological sample from the patient; and detecting the level of the antigen-presenting cell in the biological sample. In aspects, the disclosure provides methods of treating cancer in a patient in need thereof comprising: administering to the patient an effective amount of an anti-CD73 antibody; obtaining a biological sample from the patient; and detecting the level of the antigen-presenting cell in the biological sample. In aspects, the disclosure provides method of treating cancer in a patient in need thereof comprising: administering to the patient an effective amount of an anti-CD73 antibody to effectively activate an antigen-presenting cell; obtaining a biological sample from the patient; and detecting the level of the antigen-presenting cell in the biological sample. In aspects, the level of the antigen-presenting cell in the biological sample is monitored by flow cytometry, immunohistochemistry, or a combination thereof. In aspects, the antigen-presenting cell is in a cancer tumor microenvironment. In aspects, the level of the antigen-presenting cell is monitored through a biological sample from the patient, such as a blood sample, such as a peripheral blood sample. In aspects, the biological sample comprises B cells in an amount of about 0.2×10e6 cells/mL or more, as measured by flow cytometry. In aspects, the biological sample comprises B cells in an amount of about 0.3×10e6 cells/mL or more, as measured by flow cytometry. In aspects, the biological sample comprises B cells in an amount of about 0.4×10e6 cells/mL or more, as measured by flow cytometry. In aspects, the biological sample comprises B cells in an amount of about 0.5×10e6 cells/mL or more, as measured by flow cytometry. In aspects, the biological sample comprises B cells in an amount of about 0.6×10e6 cells/mL or more, as measured by flow cytometry. In aspects, the biological sample comprises dendritic cell in an amount of about 0.03×10e6 cell/mL or more, as measured by flow cytometry. In aspects, the biological sample comprises dendritic cell in an amount of about 0.04×10e6 cell/mL or more, as measured by flow cytometry. In aspects, the biological sample comprises dendritic cell in an amount of about 0.05×10e6 cell/mL or more, as measured by flow cytometry. In aspects, the biological sample comprises dendritic cell in an amount of about 0.06×10e6 cell/mL or more, as measured by flow cytometry. In aspects, the biological sample comprises dendritic cell in an amount of about 0.07×10e6 cell/mL or more, as measured by flow cytometry. In aspects, the antigen presenting cell is a B cell. In aspects, the antigen presenting cell is a B cell that is CD19+. In aspects, the antigen presenting cell is a dendritic cell. In aspects, the antigen presenting cell is a classical dendritic cell. In aspects, the antigen presenting cell is a plasmacytoid dendritic cell. In aspects, the antigen presenting cell is positive for CD3, CD14, CD19, CD69, CD83, CD86, MHC Class II (e.g., HLA-DR), BDCA-2, BDCA-4, CD11c^{low}, CD45RA, CD123, ILT-7, TLR7, TLR9, or a combination of two or more thereof. In aspects, the anti-CD73 antibody is any described herein. In aspects, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In aspects, the anti-CD73 antibody is CPI-006.

In embodiments, the disclosure provides methods of treating cancer in a patient in need thereof comprising administering to the patient an effective amount of an anti-CD73 antibody to effectively activate activation markers. In aspects, the disclosure provides methods of treating cancer in a patient in need thereof comprising: administering to the patient an effective amount of an anti-CD73 antibody; and monitoring a level of an activation marker. In aspects, the disclosure provides method of treating cancer in a patient in need thereof comprising: administering to the patient an effective amount of an anti-CD73 antibody to effectively activate an activation marker; and monitoring a level of the activation marker. In aspects, the activation marker is a B cell. In aspects, the activation marker is a dendritic cell. In aspects, the activation marker is CD69. In aspects, the activation marker is CD83. In aspects, the activation marker is CD25. In aspects, the level of the activation marker in the biological sample is monitored by flow cytometry, immunohistochemistry, or a combination thereof. In aspects, the level of the activation marker is monitored through a biological sample from the patient, such as a blood sample, such as a peripheral blood sample. In aspects, the biological sample comprises B cells in an amount of about 0.2×10e6 cells/mL or more, as measured by flow cytometry. In aspects, the biological sample comprises B cells in an amount of about 0.3×10e6 cells/mL or more, as measured by flow cytometry. In aspects, the biological sample comprises B cells in an amount of about 0.4×10e6 cells/mL or more, as measured by flow cytometry. In aspects, the biological sample comprises B cells in an amount of about 0.5×10e6 cells/mL or more, as measured by flow cytometry. In aspects, the biological sample comprises B cells in an amount of about 0.6×10e6 cells/mL or more, as measured by flow cytometry. In aspects, the biological sample comprises dendritic cell in an amount of about 0.03×10e6 cell/mL or more, as measured by flow cytometry. In aspects, the biological sample comprises dendritic cell in an amount of about 0.04×10e6 cell/mL or more, as measured by flow cytometry. In aspects, the biological sample comprises dendritic cell in an amount of about 0.05×10e6 cell/mL or more, as measured by flow cytometry. In aspects, the biological sample comprises dendritic cell in an amount of about 0.06×10e6 cell/mL or more, as measured by flow cytometry. In aspects, the biological sample comprises dendritic cell in an amount of about 0.07×10e6 cell/mL or more, as measured by flow cytometry.

In embodiments, the disclosure provides methods of treating cancer in a patient in need thereof comprising administering to the patient an effective amount of an anti-CD73 antibody to effectively activate an activation marker; obtaining a biological sample from the patient; and detecting the level of the activation marker in the biological sample. In aspects, the disclosure provides methods of treating cancer in a patient in need thereof comprising: administering to the patient an effective amount of an anti-CD73 antibody; obtaining a biological sample from the patient; and detecting the level of an activation marker in the biological sample. In aspects, the disclosure provides method of treating cancer in a patient in need thereof comprising: administering to the patient an effective amount of an anti-CD73 antibody to effectively activate an activation marker; obtaining a biological sample from the patient; and detecting the level of the activation marker in the biological sample. In aspects, the level of the activation marker in the biological sample is monitored by flow cytometry, immunohistochemistry, or a combination thereof. In aspects, the activation marker is a B cell. In aspects, the activation marker is a dendritic cell. In aspects, the activation marker is CD69. In aspects, the activation marker is CD83. In aspects, the activation marker is CD25. In aspects, the level of the activation marker in the biological sample is monitored by flow cytometry, immunohistochemistry, or a combination thereof. In aspects, the level of the activation marker is monitored through a biological sample from the patient, such as a blood sample, such as a peripheral blood sample. In aspects, the biological sample comprises B cells in an amount of about 0.2×10e6 cells/mL or more, as measured by flow cytometry. In aspects, the biological sample comprises B cells in an amount of about 0.3×10e6 cells/mL or more, as measured by flow cytometry. In aspects, the biological sample comprises B cells in an amount of about 0.4×10e6 cells/mL or more, as measured by flow cytometry. In aspects, the biological sample comprises B cells in an amount of about 0.5×10e6 cells/mL or more, as measured by flow cytometry. In aspects, the biological sample comprises B cells in an amount of about 0.6×10e6 cells/mL or more, as measured by flow cytometry. In aspects, the biological sample comprises dendritic cell in an amount of about 0.03×10e6 cell/mL or more, as measured by flow cytometry. In aspects, the biological sample comprises dendritic cell in an amount of about 0.04×10e6 cell/mL or more, as measured by flow cytometry. In aspects, the biological sample comprises dendritic cell in an amount of about 0.05×10e6 cell/mL or more, as measured by flow cytometry. In aspects, the biological sample comprises dendritic cell in an amount of about 0.06×10e6 cell/mL or more, as measured by flow cytometry. In aspects, the biological sample comprises dendritic cell in an amount of about 0.07×10e6 cell/mL or more, as measured by flow cytometry.

Dosage

The anti-CD73 inhibitor is administered to subjects in an effective amount to treat the disease in the subject. In embodiments, the effective amount is about 1 mg/kg, 3 mg/kg, 6 mg/kg, 10 mg/kg, 30 mg/kg, 40 mg/kg, or 120 mg/kg. In aspects, the effective amount is about 0.05 mg/kg, 0.1 mg/kg, 0.3 mg/kg, 1 mg/kg, 3 mg/kg, 6 mg/kg, 10 mg/kg, 30 mg/kg, 40 mg/kg, or 120 mg/kg. In aspects, the effective amount is about 0.05 mg/kg. In aspects, the effective amount is 0.05 mg/kg. In aspects, the effective amount is about 0.1 mg/kg. In aspects, the effective amount is 0.1 mg/kg. In aspects, the effective amount is about 0.3 mg/kg. In aspects, the effective amount is 0.3 mg/kg. In aspects, the effective amount is about 1 mg/kg. In aspects, the effective amount is 1 mg/kg. In aspects, the effective amount is about 3 mg/kg. In aspects, the effective amount is 3 mg/kg. In aspects, the effective amount is about 6 mg/kg. In aspects, the effective amount is 6 mg/kg. In aspects, the effective amount is about 10 mg/kg. In aspects, the effective amount is 10 mg/kg. In aspects, the effective amount is about 12 mg/kg. In aspects, the effective amount is 12 mg/kg. In aspects, the effective amount is about 30 mg/kg. In aspects, the effective amount is 30 mg/kg. In aspects, the effective amount is about 40 mg/kg. In aspects, the effective amount is 40 mg/kg. In aspects, the effective amount is about 120 mg/kg. In aspects, the effective amount is 120 mg/kg. In aspects, the effective amount administered results in serum levels of the antibody of about 10 μg/ml.

In embodiments, the anti-CD73 antibody is administered to a subject in an effective amount of at least 1 mg/kg. In aspects, the effective amount is at least 2 mg/kg. In aspects, the effective amount is at least 2 mg/kg. In aspects, the effective amount is at least 3 mg/kg. In aspects, the effective amount is at least 4 mg/kg. In aspects, the effective amount is at least 5 mg/kg. In aspects, the effective amount is at least 6 mg/kg. In aspects, the effective amount is at least 7 mg/kg. In aspects, the effective amount is at least 8 mg/kg. In aspects, the effective amount is at least 9 mg/kg. In aspects, the effective amount is at least 10 mg/kg. In aspects, the effective amount is at least 11 mg/kg. In aspects, the effective amount is at least 12 mg/kg. In aspects, the effective amount is at least 13 mg/kg. In aspects, the effective amount is at least 14 mg/kg. In aspects, the effective amount is at least 15 mg/kg. In aspects, the effective amount is from about 1 mg/kg to about 100 mg/kg. In aspects, the effective amount is from about 2 mg/kg to about 90 mg/kg. In aspects, the effective amount is from about 3 mg/kg to about 80 mg/kg. In aspects, the effective amount is from about 4 mg/kg to about 70 mg/kg. In aspects, the effective amount is from about 5 mg/kg to about 60 mg/kg. In aspects, the effective amount is from about 6 mg/kg to about 50 mg/kg. In aspects, the effective amount is from about 4 mg/kg to about 25 mg/kg. In aspects, the effective amount is from about 5 mg/kg to about 25 mg/kg. In aspects, the effective amount is from about 6 mg/kg to about 25 mg/kg. In aspects, the effective amount is from about 7 mg/kg to about 25 mg/kg. In aspects, the effective amount is from about 8 mg/kg to about 25 mg/kg. In aspects, the effective amount is from about 9 mg/kg to about 25 mg/kg. In aspects, the effective amount is from about 10 mg/kg to about 25 mg/kg. In aspects, the effective amount is from about 5 mg/kg to about 15 mg/kg. In aspects, the effective amount is from about 6 mg/kg to about 12 mg/kg. In aspects, the effective amount is about 4 mg/kg. In aspects, the effective amount is about 5 mg/kg. In aspects, the effective amount is about 6 mg/kg. In aspects, the effective amount is about 7 mg/kg. In aspects, the effective amount is about 8 mg/kg. In aspects, the effective amount is about 9 mg/kg. In aspects, the effective amount is about 10 mg/kg. In aspects, the effective amount is about 11 mg/kg. In aspects, the effective amount is about 12 mg/kg. In aspects, the effective amount is about 13 mg/kg. In aspects, the effective amount is about 14 mg/kg. In aspects, the effective amount is about 15 mg/kg. In aspects, the effective amount is about 16 mg/kg. In aspects, the effective amount is about 17 mg/kg. In aspects, the effective amount is about 18 mg/kg. In aspects, the effective amount is about 19 mg/kg. In aspects, the effective amount is about 20 mg/kg. In aspects, the effective amount is about 21 mg/kg. In aspects, the effective amount is about 22 mg/kg. In aspects, the effective amount is about 23 mg/kg. In aspects, the effective amount is about 24 mg/kg. In aspects, the effective amount is about 25 mg/kg.

In aspects, the anti-CD73 antibody is administered by parenteral injection. In aspects, the injection is a bolus injection. In aspects, the injection is an infusion (e.g., over the course of 5 minutes to 2 hours; or from about 30 minutes to about 90 minutes). In aspects, the anti-CD73 antibody is administered once per week (i.e., once every 7 days), once every two weeks (e.g., once every 14 days), once every three weeks (e.g., once every 21 days), or once per month (e.g., once every 28 days).

In embodiments for the methods provided herein the anti-CD73 antibody may be administered at a half maximal effective concentration (EC₅₀) of at least 100 nM (e.g., 100, 105, 110, 115, 120, 125, 130, 135, 140, 145, 150, 155, 160, 165, 170, 175, 180, 185, 190, 195, 200, 210, 220, 230, 240, 250 nM). In aspects, the anti-CD73 antibody is administered at a half maximal effective concentration (EC₅₀) of at least 100 nM. In aspects, the antibody is administered at a half maximal effective concentration (EC₅₀) of 110 nM. In aspects, the antibody is administered at a half maximal effective concentration (EC₅₀) of 115 nM. In aspects, the antibody is administered at a half maximal effective concentration (EC₅₀) of 120 nM. In aspects, the antibody is administered at a half maximal effective concentration (EC₅₀) of 125 nM. In aspects, the antibody is administered at a half maximal effective concentration (EC₅₀) of 130 nM. In aspects, the antibody is administered at a half maximal effective concentration (EC₅₀) of 135 nM. In aspects, the antibody is administered at a half maximal effective concentration (EC₅₀) of 140 nM. In aspects, the antibody is administered at a half maximal effective concentration (EC₅₀) of 145 nM. In aspects, the antibody is administered at a half maximal effective concentration (EC₅₀) of 150 nM. In aspects, the antibody is administered at a half maximal effective concentration (EC₅₀) of 155 nM. In aspects, the antibody is administered at a half maximal effective concentration (EC₅₀) of 160 nM. In aspects, the antibody is administered at a half maximal effective concentration (EC₅₀) of 165 nM. In aspects, the antibody is administered at a half maximal effective concentration (EC₅₀) of 170 nM. In aspects, the antibody is administered at a half maximal effective concentration (EC₅₀) of 175 nM. In aspects, the antibody is administered at a half maximal effective concentration (EC₅₀) of 180 nM. In aspects, the antibody is administered at a half maximal effective concentration (EC₅₀) of 185 nM. In aspects, the antibody is administered at a half maximal effective concentration (EC₅₀) of 190 nM. In aspects, the antibody is administered at a half maximal effective concentration (EC₅₀) of 195 nM. In aspects, the antibody is administered at a half maximal effective concentration (EC₅₀) of 200 nM. In aspects, the antibody is administered at a half maximal effective concentration (EC₅₀) of 210 nM. In aspects, the antibody is administered at a half maximal effective concentration (EC₅₀) of 220 nM. In aspects, the antibody is administered at a half maximal effective concentration (EC₅₀) of 230 nM. In aspects, the antibody is administered at a half maximal effective concentration (EC₅₀) of 240 nM. In aspects, the antibody is administered at a half maximal effective concentration (EC₅₀) of 250 nM.

In embodiments, the antibody is administered at an EC₅₀ of about 137 nM. In aspects, the antibody is administered at an EC₅₀ of 137 nM. In aspects, the antibody is administered at an EC₅₀ of about 189 nM. In aspects, the antibody is administered at an EC₅₀ of 189 nM.

Compositions

Any of the embodiments described above for anti-CD73 antibodies may be applicable to the compositions provided herein including embodiments thereof. Therefore, the anti-CD73 antibody used for the compositions provided herein may, for example, include heavy and light CDRs, wherein the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and said CDR H3 has a sequence of SEQ ID NO:6; include a humanized heavy chain variable region including the sequence of SEQ ID NO:7; may be an IgG4; may be capable of binding a CD73 antigen with an equilibrium dissociation constant (K_{D}) from about 0.3 to about 25 nM; may further include a glutamine at a position corresponding to Kabat position 297 or may form part of a cell, wherein the cell is a B cell.

In an aspect, an anticancer immunogenic composition is provided. The composition includes an anticancer immunogenic agent and an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. An “anticancer immunogenic agent” as provided herein refers to an agent having anticancer and immunogenic abilities. The term “immunogenic” is a term well known in the art and is used herein according to its conventional meaning. An immunogenic agent is an agent (e.g., compound molecule, antigen, epitope) having the ability to provoke an immune response in the body of a human or other animal. The term “immunogenicity” refers to the ability to induce a humoral and/or cell-mediated immune responses. In aspects, the anticancer immunogenic agent is an oncolytic virus or a cancer cell component.

In another aspect, an antiviral immunogenic composition is provided. The composition includes an antiviral immunogenic agent and an anti-CD73 antibody, wherein the anti-CD73 antibody includes a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

The following examples are for purposes of illustration and are not intended to limit the spirit or scope of the disclosure or claims.

### Example 1

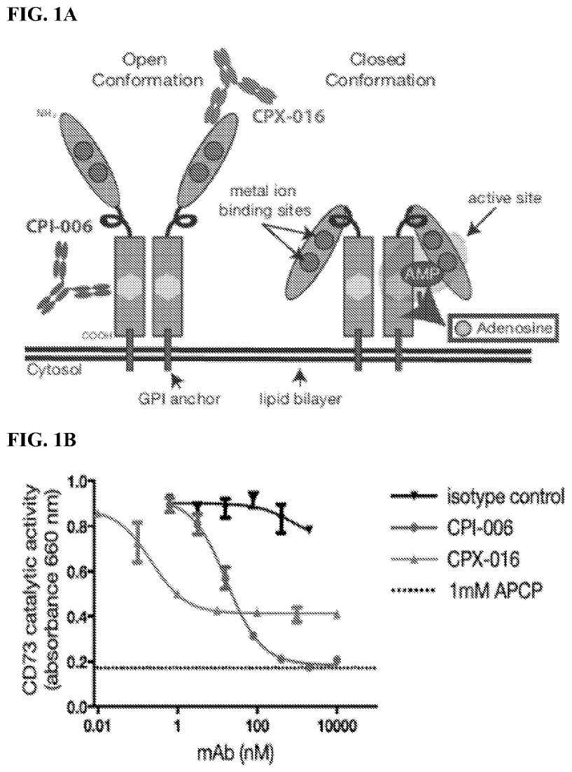

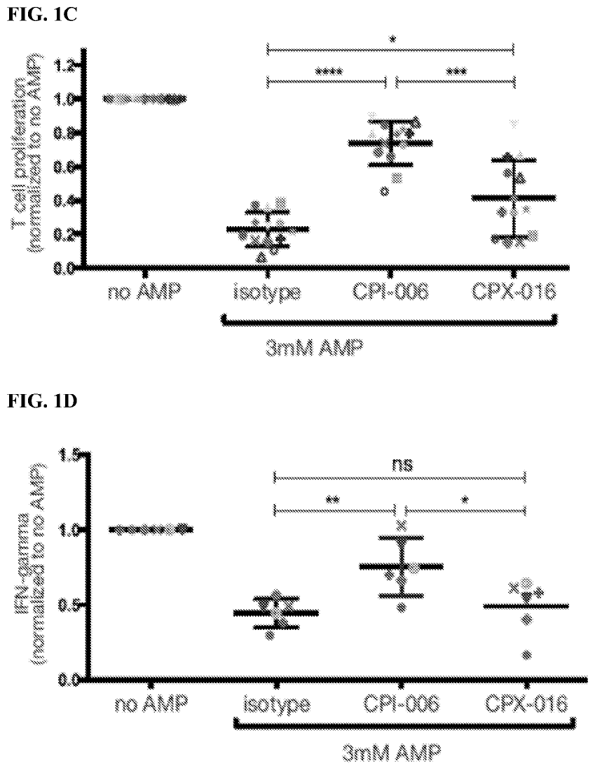

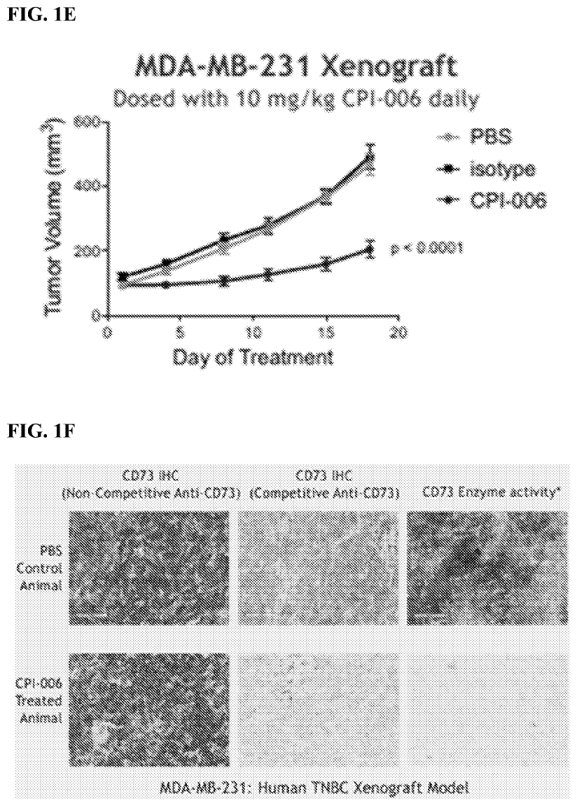

MDA-MB-231 cells (e.g., available at ATCC® as HTB-26™) were incubated with a human IgG1 isotype control, CPI-006, CPX-016, or adenosine 5′-(α,β-methylene)diphosphate (also known as APCP, which is a small molecule inhibitor of CD73 enzymatic activity), prior to addition of 250 μM of adenosine monophosphate (AMP). The CD73 catalytic activity is shown in FIG. 1B. Phosphate levels were measured in the cell culture supernatant using the Sensolyte Malachite Green assay kit. Peripheral blood mononuclear cells (PBMC) were isolated from healthy donors and labeled with CellTrace™ Violet (ThermoFisher Scientific) prior to culture with 1 μg/mL anti-CD3 and anti-CD28, 200 units/mL IL-2, 3 mM AMP and indicated treatments (CPI-006 at 500 nM; CPX-016 at 500 nM; human IgG1 isotype control at 890 nM). T cell proliferation was measured by Cell Trace Violet dilution and was defined by gating relative to unstimulated PBMC (FIG. 1C). IFN-gamma production was measured in cell culture supernatants by AlphaLISA (FIG. 1D). Results shown in FIGS. 1B-D illustrate that inhibition of CD73 activity has an agonistic effect on immunomodulatory activity.

### Example 2

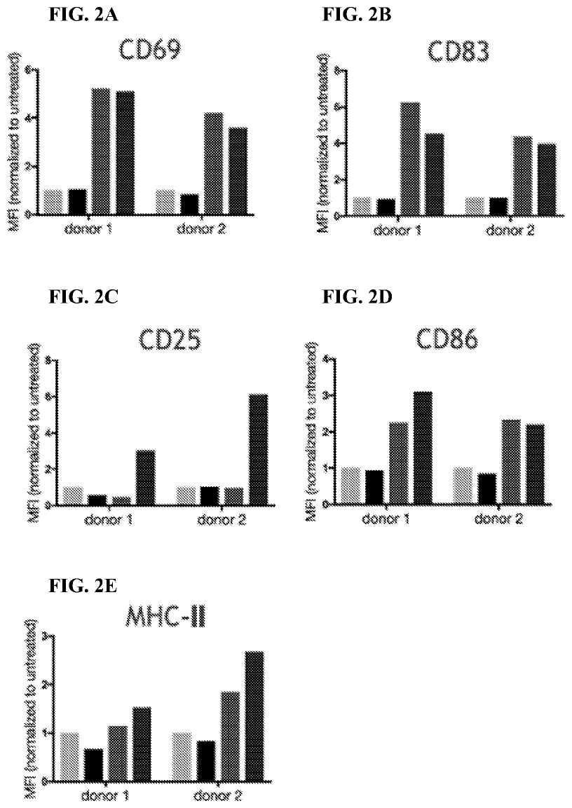

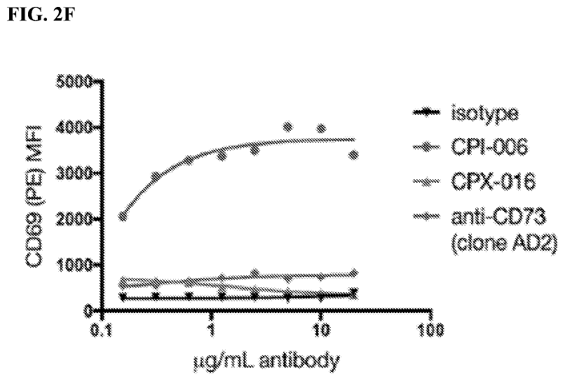

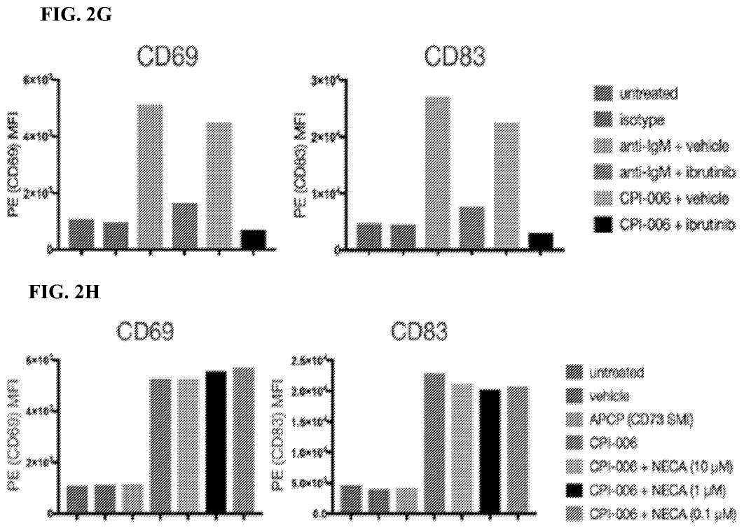

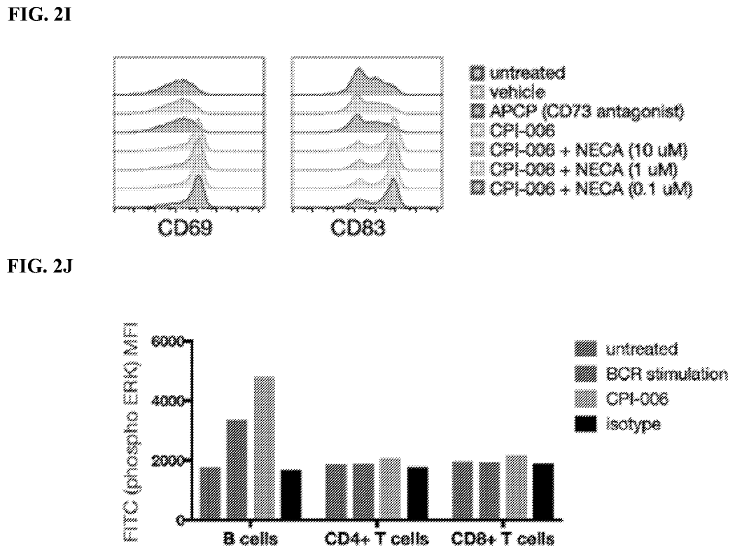

PBMC were isolated from healthy donors and incubated with bead-bound anti-IgM, CPI-006 (10 μg/mL), or human IgG1 isotype control (10 μg/mL) overnight. Flow cytometry analysis was performed with gating on B cells (CD19+CD3−) and mean fluorescence intensity (MFI) was reported for staining with antibodies to the cell surface markers CD69, CD83, and CD25, as shown in FIGS. 2A-2C. PBMC were isolated from healthy donors and incubated with CPI-006, CPI-016, human IgG1 isotype control, and anti-human CD73 antibody, clone AD2 (available from Bio-Rad Antibodies) over a range of concentrations from about 0.1 μg/mL to more than 10 μg/mL. Flow cytometry analysis was performed with gating on B cells (CD19+CD3−) and mean fluorescence intensity (MFI) is reported for antibody staining of CD69, as shown in FIG. 2F. PBMC were isolated from healthy donors and incubated overnight with bead-bound anti-IgM or CPI-006 (1 μg/mL)+/−BTK inhibitor, ibrutinib (100 nM). Flow cytometry analysis was performed with gating on B cells (CD19+CD3−) and mean fluorescence intensity (MFI) is reported for antibody staining of CD69 and CD83, as shown in FIG. 2G. PBMC were isolated from healthy donors and incubated overnight with APCP (1 μM) or CPI-006 (1 μg/mL)+/−NECA (5′-N-ethylcarboxamidoadenosine). Flow cytometry analysis was performed with gating on B cells (CD19+CD3−) and mean fluorescence intensity (MFI) is reported for antibody staining of CD69, as shown in FIGS. 2H-2I. PBMC were isolated from healthy donors and incubated with the indicated treatments for 15 minutes. Flow cytometry analysis was performed with gating on the indicated cell populations and staining of phospho ERK was evaluated, as shown in FIG. 2J. Results from the above-described studies show CPI-006 directly activates human B lymphocytes.

### Example 3

A Phase 1/1B clinical trial was designed with the protocol shown in Table 1. The primary objectives of the study were to show safety and tolerability of the active agents, and the second objectives of the study were to identify PK/PD profiles, drug efficacy, and biomarkers. Eligible patients were adults with solid tumors that had progressed on 1-5 prior therapies, that had an ECOG status of 0 or 1 adequate organ function, and that had CD73 expression (required in dose expansion study but not in dose escalation study). The adenosine gene signature was not used to select patients. The dose expansion includes treatment of non-small cell lung cancer, renal cell carcinoma, non-Hodgkin's lymphoma, and other types of cancers (e.g., bladder, prostate, pancreatic, colorectal, squamous cell cancer of the head and neck). CPI-006 was given as a one hour infusion every three weeks, and for the combination treatments 100 mg po BID of ciforadenant was administered. Biomarker assessment was conducted for the effects of CD73 expression in tumors, peripheral blood lymphocyte subsets, antibody occupancy of target, and serum cytokines. If one or more responses were observed in a disease cohort in Stage 1, then the study proceeded to Stage 2.

TABLE 1
Dose Escalation
3 + 3 Design: 1, 3, 6, 12, 18, 24 mg/kg

| | | |
|---|---|---|
| CPI-006 Alone | CPI-006 + | CPI-006 + |
| | ciforadenant | pembrolizumab |

Dose Expansion
Stage 1 (n = 11 per cohort)

| | | |
|---|---|---|
| CPI-006 alone | CPI-006 + | CPI-006 + |
| | ciforadenant | pembrolizumab |

Dose Expansion
Stage 2 (n = 17 per cohort)

The baseline demographics of the patients are shown in Table 2 below, and the adverse events are shown in Table 3 (where TEAE refers to treatment-related adverse events based on any grade 3 or 4 events, or 2 or more of all grades).

TABLE 2

| | CPI-006 | CPI-006 + ciforadenant |
|---|---|---|
| Description | (n = 12) | (n = 8) |
| Age (yrs) | 62 | 64 |
| Median (range) | (46-78) | 36-86 |
| Gender, male n (%) | 10 (83%) | 8 (100%) |
| Number of prior therapies median | 4 | 4 |
| Number of prior therapies range | (1-5) | (3-7) |
| Histologies | N | N |
| Colorectal | 2 | 2 |
| Prostate | 3 | 1 |
| Pancreatic | 2 | 2 |
| Head and Neck | 2 | 1 |
| Bladder | 1 | 0 |
| Renal cell carcinoma | 1 | 2 |
| Sarcoma | 1 | 0 |

TABLE 3

| Adverse Events | CPI-006 Monotherapy | | CPI-006 + Ciforadenant | |
|---|---|---|---|---|
| Number of | (n = 12) | | (n = 8) | |
| Patients | All Grades | Grade 3 or 4 | All Grades | Grade 3 or 4 |
| Subjects with any | 8 | 1 | 5 | 0 |
| TEAE | | | | |
| Anemia | 1 | 1 | 1 | 0 |
| Diarrhea | 1 | 0 | 1 | 0 |
| Nausea | 3 | 0 | 2 | 0 |
| Chills | 4 | 0 | 1 | 0 |
| Fatigue | 2 | 0 | 2 | 0 |
| Infusion-related | 2 | 0 | 1 | 0 |
| reaction | | | | |
| Headache | 2 | 0 | 1 | 0 |
| Pruritus | 2 | 0 | 0 | 0 |

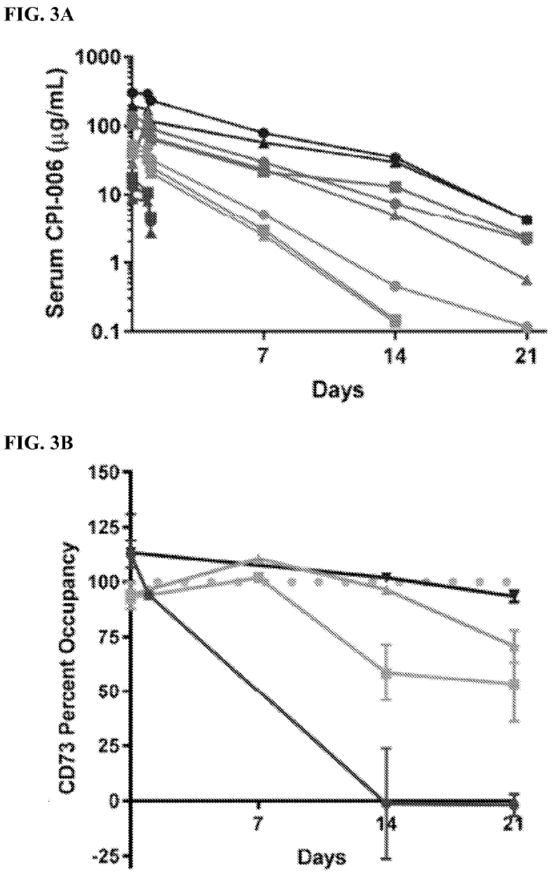

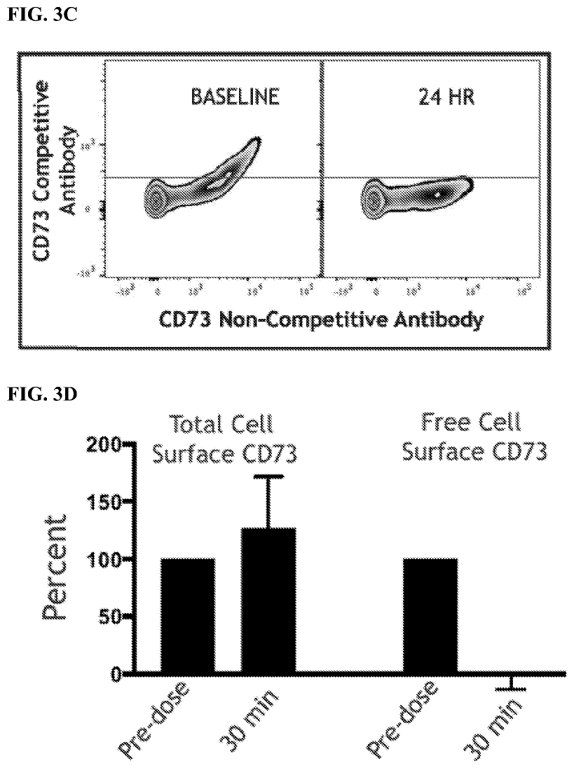

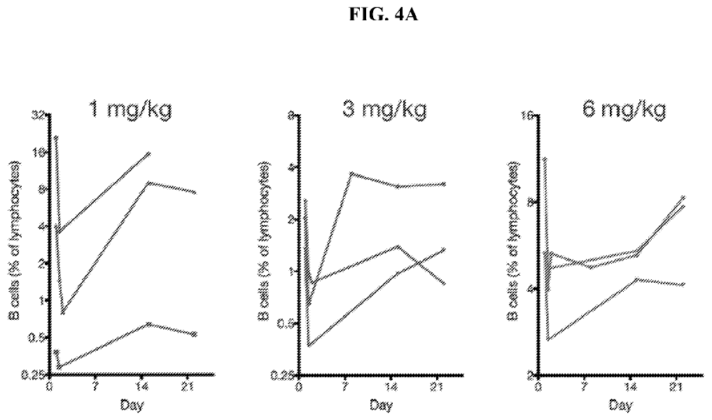

With reference to Table 1, serum samples were collected from patients treated with CPI-006 alone (i.e., monotherapy) and levels of free CPI-006 were measured by ELISA, as shown in FIG. 3A. Whole blood samples from patients treated with CPI-006 alone were fixed and receptor occupancy was measured by flow cytometry gating on CD73+CD8 T cells, as shown in FIG. 3B. As illustrated in FIGS. 3A-3D, receptor exposure to CPI-006 increases and clearance decreases with increasing doses of CPI-006, and CIP-006 is detectable for 21 days after administration of a single dose of 6 mg/kg or higher. Further, total cell surface CD73 remains unchanged, and the CPI-006 binding site is occupied. Other results from the study are described below and in Table 4. BLQ is below limit of quantification. TBD is to be determined. No grade 3/4 adverse events were reported.

TABLE 4

| | | | Occupancy of | % Decrease | Serum |
|---|---|---|---|---|---|
| | | | Peripheral | of Peripheral | CPI-006 |
| | | | B cells | B cells | (μg/mL) |
| Patient | Dose | Cancer | at CD15 | at C1D1 (0.5 h) | at C1D8 |
| 1 | 1 mg/kg | Bladder | −1.35% | 24.6% | BLQ |
| 2 | 1 mg/kg | Prostate | 65.83% | 82.6% | BLQ |
| 3 | 1 mg/kg | SCHN | 22.96% | 63.7% | BLQ |
| 4 | 3 mg/kg | Pancreatic | 88.12% | 71.2% | 2.461 |
| 5 | 3 mg/kg | Pancreatic | 74.57% | 62.3% | 5.032 |
| 6 | 3 mg/kg | Prostate | 58.19% | 68.1% | 2.937 |
| 7 | 6 mg/kg | Colorectal | 97.90% | 49.3% | 23.60 |
| 8 | 6 mg/kg | Prostate | 98.31% | 64.6% | TBD |
| 9 | 6 mg/kg | SCHN | 101.60% | 11.1% | TBE |

A tumor biopsy taken from a colorectal cancer patient treated with CPI-006 was evaluated for inhibition of CD73 enzymatic activity. Results shown in FIG. 5 illustrate CD73 is present on tumor cells, and that CPI-006 binds and saturates CD73. Further, CPI-006 binding was shown to inhibit CD73 enzymatic activity, confirming CPI-006 functions in vivo.

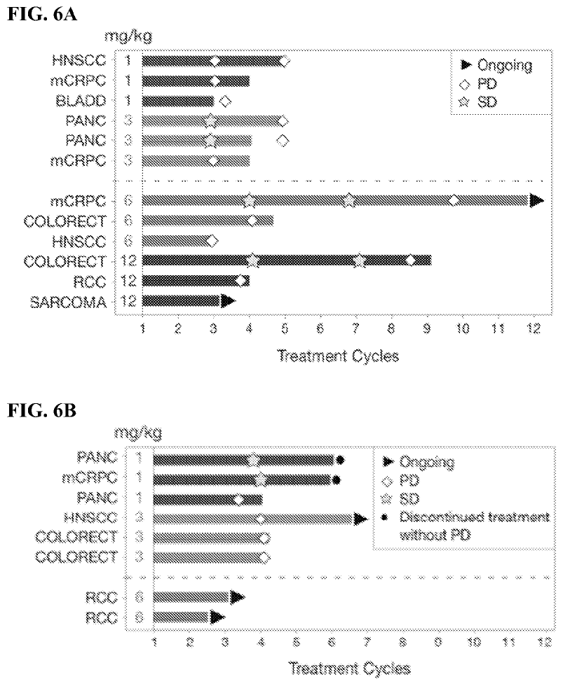

Cancer patients treated with CPI-006 monotherapy or a combination therapy of CPI-006 and ciforadenant were assessed for disease control. As shown in FIG. 6A, higher doses of CPI-006 monotherapy provide longer term disease control. Further, the combination therapy also improves disease control, as illustrated in FIG. 6B.

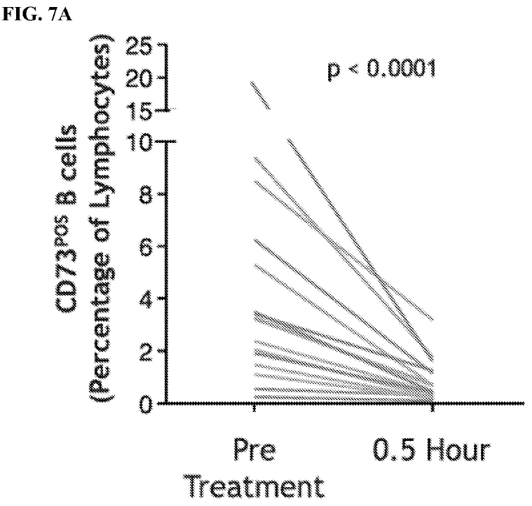

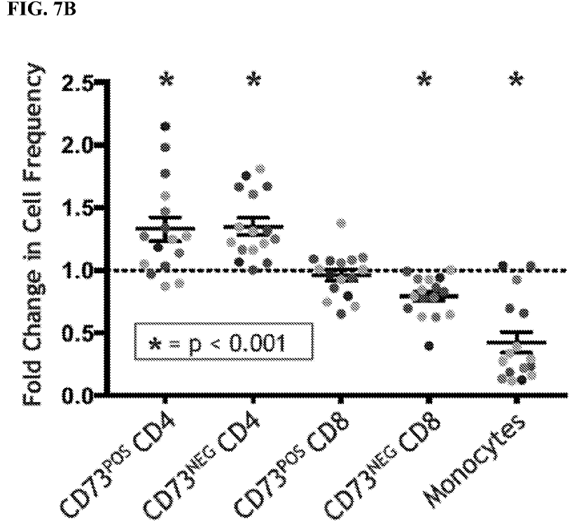

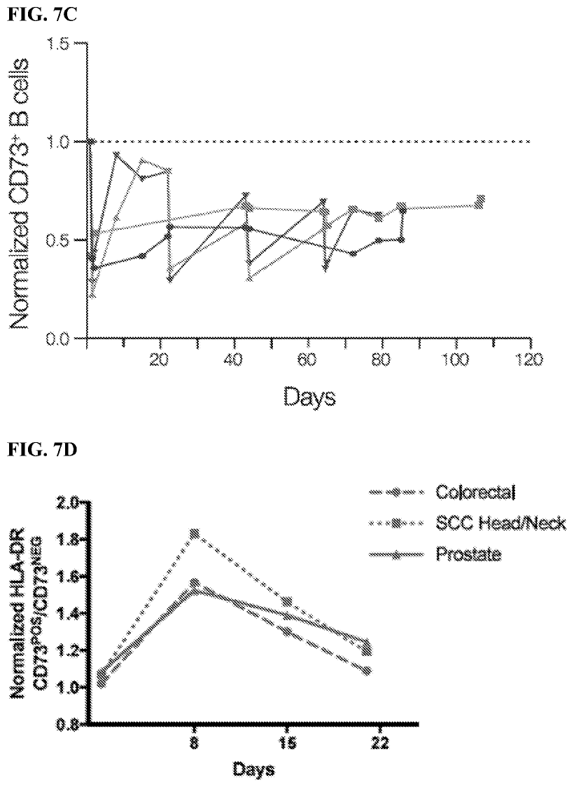

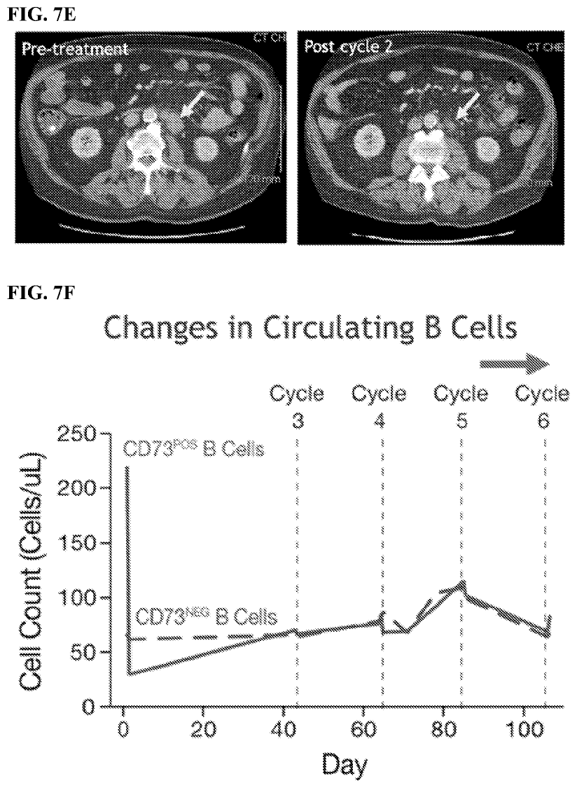

Changes in B cells and T cells were evaluated upon treatment with treatment with CPI-006. Treatment with CPI-006 show results consistent with trafficking of CD73^{POS} B cells out of the blood, redistribution of T cells (CD73^{NEG}) and monocytes, and an increase in the CD4/CD8 ratios, including CD73^{NEG} subsets, as shown in FIGS. 7A-7B. Further, as illustrated in FIG. 7C, CD73^{POS} B cells levels decrease with each infusion of CPI-006, and then partially return to a new steady state level. These results are consistent with redistribution of B cells to lymphoid tissue. No significant changes occurred in CD73^{NEG} B cells following treatment. Results in FIG. 7D demonstrate an increase in HLA-DR expression, followed by a gradual decrease in HLA-DR levels. In a patient receiving 6 mg/mkg CPI-006 monotherapy, a decrease in the target legion was observed with treatment ongoing through 11 cycles as illustrated in FIG. 7E. FIG. 7F shows the same patient receiving the 6 mg/mkg CPI-006 monotherapy had a decrease in circulating CD73^{POS} B cells following treatment. These results demonstrate CPI-006 lowers CD74^{POS} B cell levels in patients, and patients receiving CPI-006 show favorable response criteria, including decreased target legions.

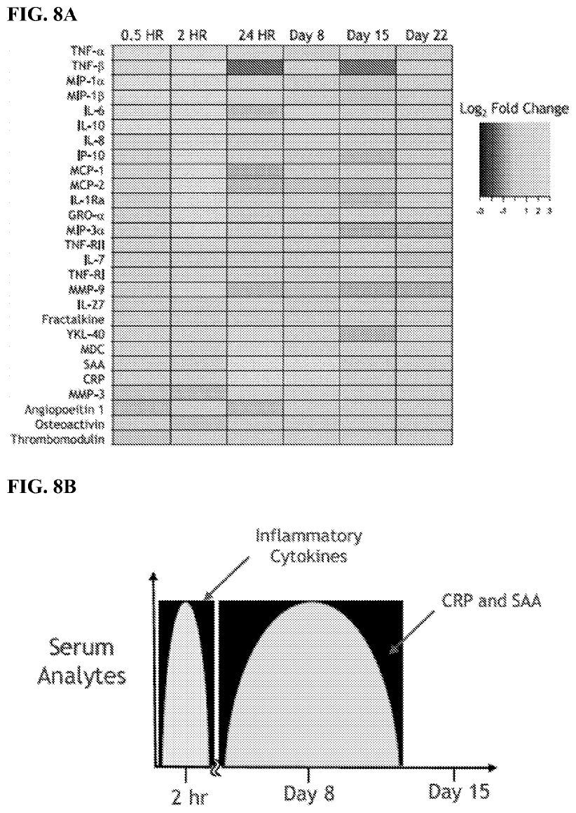

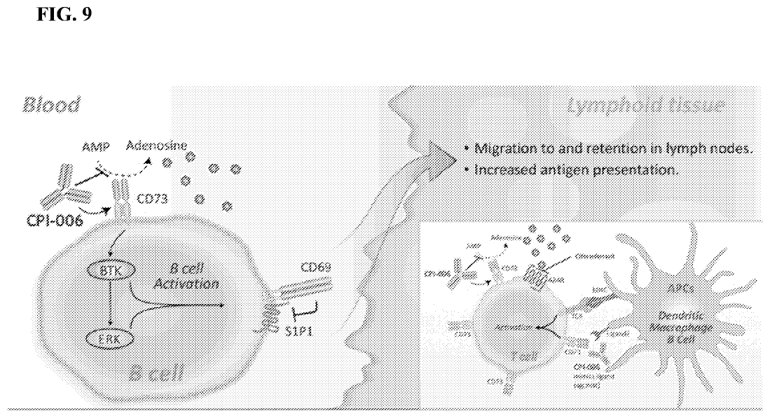

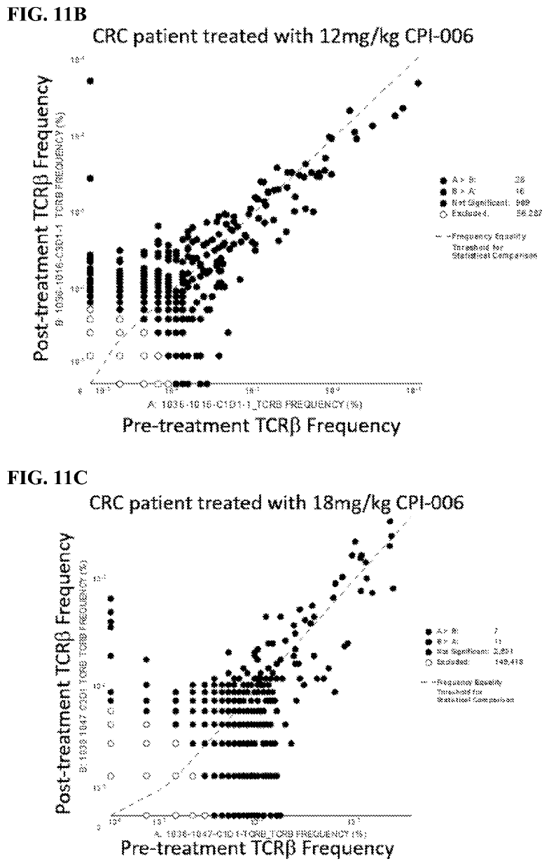

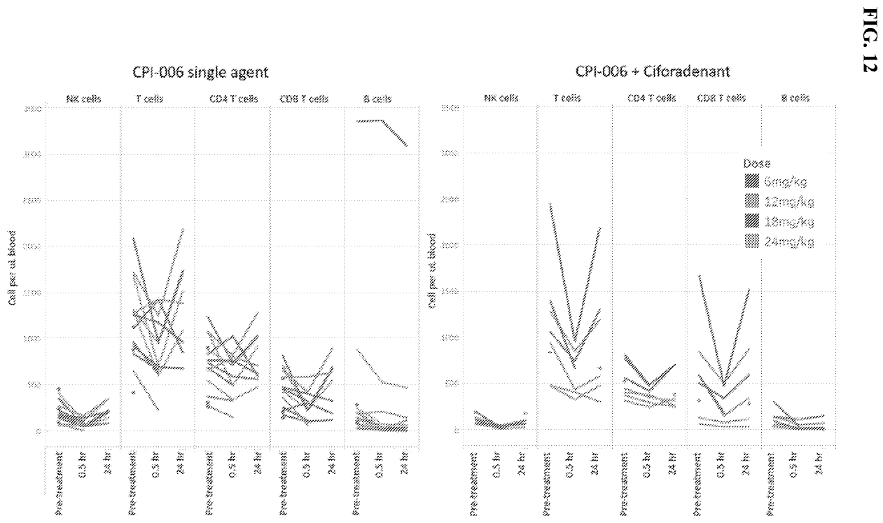

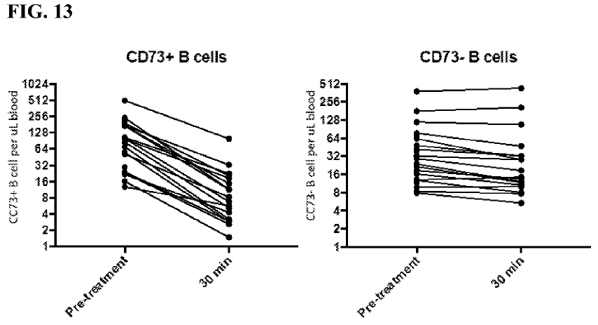

Immunomodulatory activity of CPI-006 was evaluated in patients administered CPI-006. Changes in cytokine levels in patients were measured following CPI-006 treatment, as shown in FIGS. 8A-8B. The rapid induction of cytokines including TNF-α, TNF-β, MIP-1α, MIP-1β, IL-6, IL-10, IL-8, IP-10, MCP-1, MCP-2, IL-1Ra, GRO-α, MIP-3α, TNF-RII, IL-7, MMP-9, and the subsequent increase in levels of CRP and SAA are consistent with an early inflammatory response. These results indicate CP1-006 blocks CD73 production of adenosine, and thus modulates the immune response. Further, T cell activation was evaluated upon treatment with various dosages of the monotherapy CPI-006 or a combination therapy of CPI-006 and ciforadenant. Once a T cell clone is activated, the clone can expand and produce clonal copies of the original clone. As shown in FIGS. 11A-11C, new T cell clones were generated upon treatment with the mono and combination therapies. The results further affirm CPI-006 is an immunomodulator of CD73 positive cells.

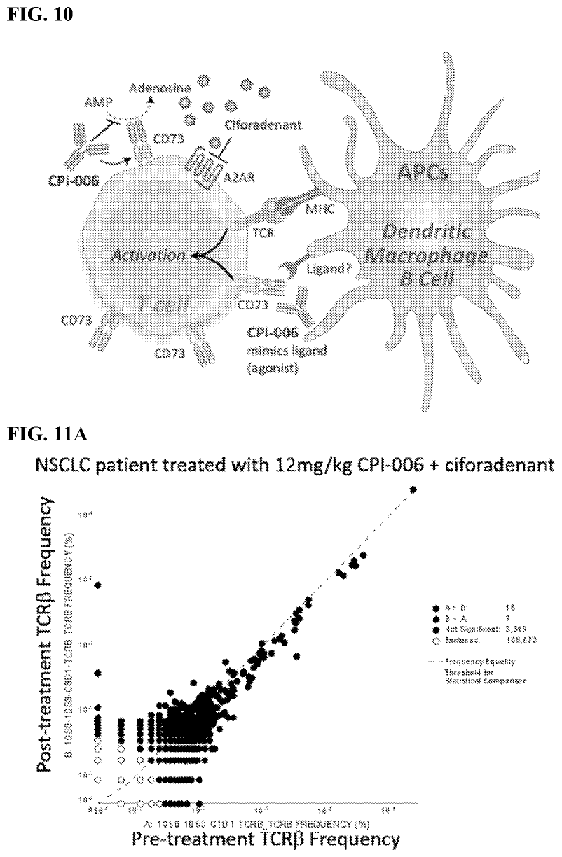

Without being bound by scientific theory, FIG. 10 shows a schematic of the agonistic immunomodulatory effects of CPI-006 with Ciforadenant. Adenosine in the tumor microenvironment is immunosuppressive. CD73 is an ectoenzyme present on many tissues including subsets of T and B cells, where CD73 converts AMP to adenosine and functions in lymphocyte adhesion, migration, and activation. CPI-006 is a humanized IgG1 Fcy receptor-deficient anti-CD73 with unique properties, such as blocking catalytic activity, and exerting agonistic immunomodulatory activity on CD73 positive cells. Ciforadenant is an adenosine A2A receptor antagonist with anti-tumor activity in animals and human clinical trials, where the adenosine signature in the tumor correlates with the adenosine response.

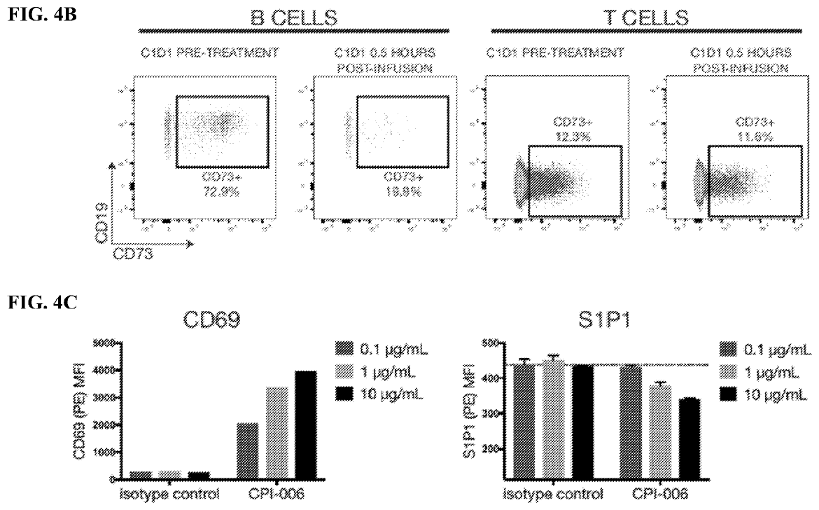

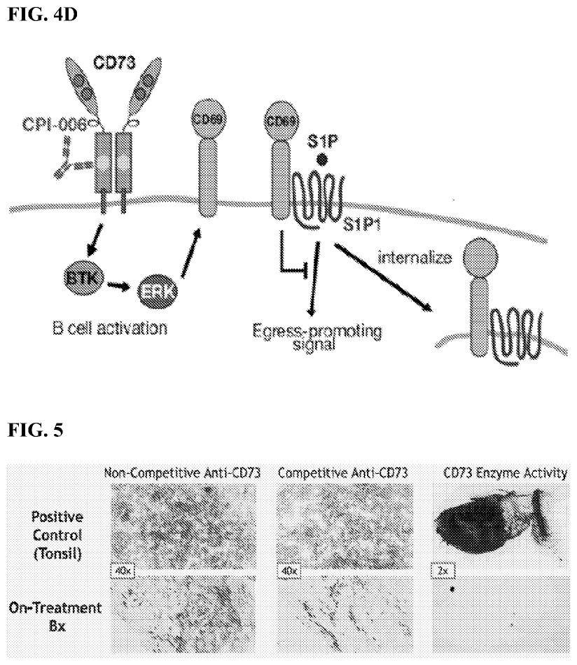

Whole blood samples from the patients treated with a single dose (i.e., 1 mg/kg, 3 mg/kg, or 6 mg/kg) of CPI-006 monotherapy were analyzed by flow cytometry to further evaluate immunomodulatory effects. Levels of B cells (CD19+CD3−) are reported as a percent of total lymphocytes (gated based on scatter properties), as shown in FIG. 4A. CD73 expression was evaluated with a non-competing anti-CD73 antibody and is reported on B cells (CD19+CD3−) and T cells (CD19−CD3+), as shown in FIG. 4B. Purified B cells from healthy human donors were incubated with CPI-006 or human IgG1 isotype control at concentrations of 0.1 μg/mL, 1 μg/mL, and 10 μg/mL for 30 minutes. Surface levels of CD69 and S1P1 were determined by flow cytometry with gating on CD73+ B cells, as shown in FIG. 4C.

Without intending to be bound by any theory of the invention, FIG. 4D provide a model for the mechanism leading to the reduction in levels of peripheral B cells by CPI-006. CPI-006 binding to CD73+ B cells induces expression of CD69, which promotes internalization of S1P1. Internalization of S1P1 promotes retention of lymphocytes in lymphoid organs. Shiow et al, Nature (2006) Vol 440, 540-544. S1P is abundant in the blood, and peripheral lymphocytes have low levels of its receptor S1P1 on their surfaces. S1P1 is upregulated on lymphocytes after migration into lymphoid organs. S1P1 signaling mediates egress of lymphocytes from lymphoid tissues, and surface expression of S1P1 is required for signaling that promotes egress. In contrast, downmodulation of S1P1 blocks egress from lymphoid organs.

Conclusions. Based on the data shown herein, it can be seen that CPI-006 targets a novel epitope on CD73; blocks production of adenosine by inhibiting the enzymatic active site; activates B cells, which leads to increased expression of CD69. The clinical data indicates that CPI-006 is well tolerated at doses of 1, 3, and 6 mg/kg with no dose-limiting toxicity; dose proportional PK and receptor occupancy was observed; and CPI-006 affects B lymphocyte trafficking as shown by transient redistribution of B cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING
SEQ ID NO: 1:
RASKNVSTSGYSYMH
SEQ ID NO: 2:
LASNLES
SEQ ID NO: 3:
QHSRELPFT
SEQ ID NO: 4:
GYTFTSYWIT
SEQ ID NO: 5:
PGSGNTNYNEKFKT
SEQ ID NO: 6:
EGGLTTEDYALDY
SEQ ID NO: 7:
QVQLVQSGAEVEKPGASVKVSCKASGYTFTSYWITWVRQAPGQGLEWMGDI
YPGSGNTNYNEKFKTRVTITADKSTSTAYMELSSLRSEDTAVYYCAKEGGL
TTEDYALDYWGQGTLVTV
SEQ ID NO: 8:
EIVLTQSPATLSLSPGERATLSCRASKNVSTSGYSYMHWYQQKPGQAPRLL
IYLASNLESGIPPRFSGSGYGTDFTLTINNIESEDAAYYFCQHSRELPFTF
GQGTKVEIK
SEQ ID NO: 9
MEWSWVFLFFLSVTTGVHSQVQLVQSGAEVEKPGASVKVSCKASGYTFTSY
WITWVRQAPGQGLEWMGDIYPGSGNTNYNEKFKTRVTITADKSTSTAYMEL
SSLRSEDTAVYYCAKEGGLTTEDYALDYWGQGTLVTVSSASTKGPSVFPLA
PSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGV
EVHNAKTKPREEQYQSTYRVVSVLTVLHQDWLNGKEYKCKVSNKALPAPIE
KTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNH
YTQKSLSLSPGK
SEQ ID NO: 10
MSVPTQVLGLLLLWLTDARCEIVLTQSPATLSLSPGERATLSCRASKNVST
SGYSYMHWYQQKPGQAPRLLIYLASNLESGIPPRFSGSGYGTDFTLTINNI
ESEDAAYYFCQHSRELPFTFGQGTKVEIKRTVAAPSVFIFPPSDEQLKSGT
ASVVCLLNNFYPREAKVQWKVDNALQSGNSQESVTEQDSKDSTYSLSSTLT
LSKADYEKHKVYACEVTHQGLSSPVTKSFNRGEC

### EMBODIMENTS

The present disclosure further provides the following embodiments:

Embodiment 1. A method of immunostimulating a subject, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 2. A method of activating B cells in a subject, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 3. A method of decreasing egress of B cells from lymphoid tissue in a subject relative to a standard control, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 4. A method of increasing retention of B cells in lymphoid organs in a subject relative to a standard control, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 5. A method of increasing internalization of a Sphingosine-1-phosphate receptor 1 (S1PR1) in a cell in a subject relative to a standard control, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 6. The method of one of Embodiments 1-5, wherein said subject is a subject having a cancer.

Embodiment 7. The method of one of Embodiments 1-5, wherein said subject is immune deficient

Embodiment 8. The method of one of Embodiments 1-5, wherein said subject has an immune deficiency disease or an autoimmune disease.

Embodiment 9. A method of treating a Sphingosine-1-phosphate receptor 1 (S1PR1)-associated disease in a subject in need thereof, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 10. The method of Embodiment 9, wherein said S1PR1-associated disease is a graft versus host disease, an autoimmune disease, an inflammatory disease, viral myocarditis, a viral disease caused by viral myocarditis, a demyelinating disease, or organ or tissue transplant rejection.

Embodiment 11. The method of Embodiment 9, wherein said S1PR1-associated disease is an autoimmune disease.

Embodiment 12. The method of Embodiment 10 or 11, wherein said S1PR1-associated disease is multiple sclerosis, rheumatoid arthritis, atopic eczema (atopic dermatitis), Behçet's disease, uvea diseases, systemic lupus erythematosus, Sjogren's syndrome, polysclerosis, myasthenia gravis, diabetes type I, endocrine eye disorders, primary biliary cirrhosis, Crohn's disease, glomerulonephritis, sarcoidosis, psoriasis, pemphigus, aplastic anemia, idiopathic thrombocytopenic purpura, allergy, polyarteritis nodosa, progressive systemic sclerosis, mixed connective-tissue disease, aortitis syndrome, polymyositis, dermatomyositis, Wegener's granulomatosis, ulcerative colitis, active chronic hepatitis, autoimmune hemolytic anemia, Evans syndrome, bronchial asthma, liver failure, renal failure, or pollinosis.

Embodiment 13. A method of treating an infectious disease in a subject in need thereof, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 14. A method of treating an inflammatory disease in a subject in need thereof, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 15. A method of treating an autoimmune disease in a subject in need thereof, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 16. The method of any one of Embodiments 1-15, wherein said anti-CD73 antibody is administered at a half maximal effective concentration (EC₅₀) of at least 100 nM.

Embodiment 17. The method of any one of Embodiments 1-15, wherein said anti-CD73 antibody is administered at an EC50 of about 137 nM.

Embodiment 18. The method of any one of Embodiments 1-15, wherein said anti-CD73 antibody is administered at an EC₅₀ of about 189 nM.

Embodiment 19. The method of any one of Embodiments 1-18, wherein said effective amount is about 1 mg/kg, 3 mg/kg, 6 mg/kg, 10 mg/kg, 30 mg/kg, 40 mg/kg, or 120 mg/kg.

Embodiment 20. The method of any one of Embodiments 1-19, wherein said CDR L1 has a sequence of SEQ ID NO:1, said CDR L2 has a sequence of SEQ ID NO:2, said CDR L3 has a sequence of SEQ ID NO:3; said CDR H1 has a sequence of SEQ ID NO:4, said CDR H2 has a sequence of SEQ ID NO:5, and said CDR H3 has a sequence of SEQ ID NO:6.

Embodiment 21. The method of any one of Embodiments 1-20, wherein the anti-CD73 antibody comprises a humanized light chain variable region and a humanized heavy chain variable region,

wherein said humanized light chain variable region comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87; and

wherein said humanized heavy chain variable region comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

Embodiment 22. The method of Embodiment 21, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

Embodiment 23. The method of Embodiment 21 or 22, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

Embodiment 24. The method of any one of Embodiments 21-23, wherein said humanized heavy chain variable region comprises the sequence of SEQ ID NO:7.

Embodiment 25. The method of any one of Embodiments 21-24, wherein said humanized light chain variable region comprises the sequence of SEQ ID NO:8.

Embodiment 26. The method of any one of Embodiments 1-25, wherein said anti-CD73 antibody is an IgG.

Embodiment 27. The method of any one of Embodiments 1-26, wherein said anti-CD73 antibody is an IgG1.

Embodiment 28. The method of any one of Embodiments 1-26, wherein said anti-CD73 antibody is an IgG4.

Embodiment 29. The method of any one of Embodiments 1-25, wherein said anti-CD73 antibody is a Fab′ fragment.

Embodiment 30. The method of any one of Embodiments 1-25, wherein said anti-CD73 antibody is a single chain antibody (scFv).

Embodiment 31. The method of any one of Embodiments 1-30, wherein said anti-CD73 antibody is capable of binding a CD73 antigen with an equilibrium dissociation constant (K_{D}) from about 0.3 to about 25 nM.

Embodiment 32. The method of any one of Embodiments 1-31, wherein said anti-CD73 antibody is capable of binding a CD73 antigen with an equilibrium dissociation constant (K_{D}) of about 0.64 nM.

Embodiment 33. The method of any one of Embodiments 1-32, wherein said anti-CD73 antibody is capable of binding a CD73 antigen at a pH of less than about 7.5.

Embodiment 34. The method of any one of Embodiments 1-33, wherein said anti anti-CD73 antibody body is capable of binding a CD73 antigen at a pH from about 6.0 to about 7.0.

Embodiment 35. The method of any one of Embodiments 1-34, wherein said anti-CD73 antibody is capable of binding a CD73 antigen at a pH of about 6.3.

Embodiment 36. The method of any one of Embodiments 1-35, wherein said anti-CD73 antibody further comprises a glutamine at a position corresponding to Kabat position 297.

Embodiment 37. The method of any one of Embodiments 1-35, wherein said anti-CD73 antibody is bound to a CD73 antigen.

Embodiment 38. The method of Embodiment 37, wherein said CD73 antigen forms part of a cell.

Embodiment 39. The method of Embodiment 38, wherein said cell is a lymphoid cell.

Embodiment 40. The method of Embodiment 38, wherein said cell is a B cell.

Embodiment 41. An anticancer immunogenic composition comprising an anticancer immunogenic agent and an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 42. The anticancer immunogenic composition of Embodiment 41, wherein said anticancer immunogenic agent is an oncolytic virus or a cancer cell component.

Embodiment 43. An antiviral immunogenic composition comprising an antiviral immunogenic agent and an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 44. The composition of any one of Embodiments 41-43, wherein said CDR L1 has a sequence of SEQ ID NO:1, said CDR L2 has a sequence of SEQ ID NO:2, said CDR L3 has a sequence of SEQ ID NO:3; said CDR H1 has a sequence of SEQ ID NO:4, said CDR H2 has a sequence of SEQ ID NO:5, and said CDR H3 has a sequence of SEQ ID NO:6.

Embodiment 45. The composition of any one of Embodiments 41-44, wherein the anti-CD73 antibody comprises a humanized light chain variable region and a humanized heavy chain variable region,

wherein said humanized light chain variable region comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87; and

wherein said humanized heavy chain variable region comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

Embodiment 46. The composition of Embodiment 45, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

Embodiment 47. The composition of Embodiment 45 or 46, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

Embodiment 48. The composition of any one of Embodiments 45-47, wherein said humanized heavy chain variable region comprises the sequence of SEQ ID NO:7.

Embodiment 49. The composition of any one of Embodiments 45-48, wherein said humanized light chain variable region comprises the sequence of SEQ ID NO:8.

Embodiment 50. The composition of any one of Embodiments 41-49, wherein said anti-CD73 antibody is an IgG.

Embodiment 51. The composition of any one of Embodiments 41-50, wherein said anti-CD73 antibody is an IgG1.

Embodiment 52. The composition of any one of Embodiments 41-50, wherein said anti-CD73 antibody is an IgG4.

Embodiment 53. The composition of any one of Embodiments 41-49, wherein said anti-CD73 antibody is a Fab′ fragment.

Embodiment 54. The composition of any one of Embodiments 41-49, wherein said anti-CD73 antibody is a single chain antibody (scFv).

Embodiment 55. The composition of any one of Embodiments 41-54, wherein said anti-CD73 antibody is capable of binding a CD73 antigen with an equilibrium dissociation constant (K_{D}) from about 0.3 to about 25 nM.

Embodiment 56. The composition of any one of Embodiments 41-55, wherein said anti-CD73 antibody is capable of binding a CD73 antigen with an equilibrium dissociation constant (K_{D}) of about 0.64 nM.

Embodiment 57. The composition of any one of Embodiments 41-56, wherein said anti-CD73 antibody is capable of binding a CD73 antigen at a pH of less than about 7.5.

Embodiment 58. The composition of any one of Embodiments 41-57, wherein said anti anti-CD73 antibody body is capable of binding a CD73 antigen at a pH from about 6.0 to about 7.0.

Embodiment 59. The composition of any one of Embodiments 41-58, wherein said anti-CD73 antibody is capable of binding a CD73 antigen at a pH of about 6.3.

Embodiment 60. The composition of any one of Embodiments 41-59, wherein said anti-CD73 antibody further comprises a glutamine at a position corresponding to Kabat position 297.

Embodiment 61. The composition of any one of Embodiments 41-60, wherein said anti-CD73 antibody is bound to a CD73 antigen.

Embodiment 62. The composition of Embodiment 61, wherein said CD73 antigen forms part of a cell.

Embodiment 63. The composition of Embodiment 62, wherein said cell is a lymphoid cell.

Embodiment 64. The composition of Embodiment 62, wherein said cell is a B cell.

Embodiment 65. A method of treating cancer in a patient in need thereof, the method comprising administering to the patient an effective amount of an anti-CD73 antibody to effectively activate an antigen-presenting cell.

Embodiment 66. A method of treating cancer in a patient in need thereof, the method comprising: (i) administering to the patient an effective amount of an anti-CD73 antibody; and (ii) monitoring a level of an antigen-presenting cell.

Embodiment 67. A method of treating cancer in a patient in need thereof, the method comprising: (i) administering to the patient an effective amount of an anti-CD73 antibody to effectively activate an antigen-presenting cell; and (ii) monitoring a level of the antigen-presenting cell.

Embodiment 68. The method of Embodiment 66 or 67, wherein monitoring the level of the antigen-presenting cell comprises (a) obtaining a biological sample from the patient, and (b) detecting the level of the antigen-presenting cell in the biological sample.

Embodiment 69. The method of Embodiment 68, wherein the biological sample is a blood sample.

Embodiment 70. The method of Embodiment 68, wherein the biological sample is a peripheral blood sample.

Embodiment 71. The method of any one of Embodiments 68 to 70, wherein detecting the level of the antigen-presenting cell in the biological sample comprises flow cytometry, immunohistochemistry, or a combination thereof.

Embodiment 72. The method of any one of Embodiments 65 to 71, wherein the antigen-presenting cell is in a cancer tumor microenvironment.

Embodiment 73. The method of any one of Embodiments 65 to 72, wherein the antigen-presenting cell is a B cell.

Embodiment 74. The method of Embodiment 73, wherein the B cell is CD19+.

Embodiment 75. The method of Embodiment 73 or 74, wherein the B cell is present in the biological sample in an amount of about 0.5×10e6 cells/mL or more.

Embodiment 76. The method of any one of Embodiments 65 to 72, wherein the antigen-presenting cell is a dendritic cell.

Embodiment 77. The method of Embodiment 76, wherein the dendritic cell is a classical dendritic cell.

Embodiment 78. The method of Embodiment 76, wherein the dendritic cell is a plasmacytoid dendritic cell.

Embodiment 79. The method of Embodiment 76, wherein the dendritic cell is positive for CD3, CD14, CD19, or a combination thereof.

Embodiment 80. The method of Embodiment 76, wherein the dendritic cell is positive for CD86.

Embodiment 81. The method of Embodiment 76, wherein the dendritic cell is positive for MHC Class II.

Embodiment 82. The method of any one of Embodiments 76 to 81, wherein the dendritic cell is positive for BDCA-2, BDCA-4, CD11c^{low}, CD45RA, CD123, ILT-7, TLR7, TLR9, or a combination of two or more thereof.

Embodiment 83. The method of any one of Embodiments 76 to 82, wherein the dendritic cell is present in the biological sample in an amount of about 0.06×10e6 cell/mL or more.

Embodiment 84. The method of any one of Embodiments 65 to 72, wherein the antigen-presenting cell comprises CD69, CD83, or a combination thereof.

Embodiment 85. The method of any one of Embodiments 65 to 84, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 86. The method of Embodiment 85, wherein said CDR L1 has a sequence of SEQ ID NO:1, said CDR L2 has a sequence of SEQ ID NO:2, said CDR L3 has a sequence of SEQ ID NO:3; said CDR H1 has a sequence of SEQ ID NO:4, said CDR H2 has a sequence of SEQ ID NO:5, and said CDR H3 has a sequence of SEQ ID NO:6.

Embodiment 87. The method of any one of Embodiments 65 to 84, wherein the anti-CD73 antibody comprises a humanized light chain variable region and a humanized heavy chain variable region; wherein said humanized light chain variable region comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87; and wherein said humanized heavy chain variable region comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

Embodiment 88. The method of Embodiment 87, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

Embodiment 89. The method of Embodiment 87 or 88, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

Embodiment 90. The method of any one of Embodiments 65 to 84, wherein said humanized heavy chain variable region comprises the sequence of SEQ ID NO:7.

Embodiment 91. The method of any one of Embodiments 65 to 84, wherein said humanized light chain variable region comprises the sequence of SEQ ID NO:8.

Embodiment 92. The method of any one of Embodiments 65 to 84, wherein said humanized heavy chain variable region comprises the sequence of SEQ ID NO:7, and said humanized light chain variable region comprises the sequence of SEQ ID NO:8.

Embodiment 93. The method of any one of Embodiments 65 to 84, wherein said anti-CD73 antibody is an IgG.

Embodiment 94. The method of any one of Embodiments 65 to 84, wherein said anti-CD73 antibody is an IgG1.

Embodiment 95. The method of any one of Embodiments 65 to 84, wherein said anti-CD73 antibody is an IgG4.

Embodiment 96. The method of any one of Embodiments 65 to 84, wherein said anti-CD73 antibody is a Fab′ fragment.

Embodiment 97. The method of any one of Embodiments 65 to 84, wherein said anti-CD73 antibody is a single chain antibody (scFv).

Embodiment 98. The method of any one of Embodiments 65 to 97, wherein said anti-CD73 antibody is capable of binding a CD73 antigen with an equilibrium dissociation constant (K_{D}) from about 0.3 to about 25 nM.

Embodiment 99. The method of Embodiment 99, wherein said anti-CD73 antibody is capable of binding a CD73 antigen with an equilibrium dissociation constant (K_{D}) of about 0.64 nM.

Embodiment 100. The method of any one of Embodiments 65 to 99, wherein said anti-CD73 antibody is capable of binding a CD73 antigen at a pH of less than about 7.5.

Embodiment 101. The method of Embodiment 100, wherein said anti-CD73 antibody body is capable of binding a CD73 antigen at a pH from about 6.0 to about 7.0.

Embodiment 102. The method of Embodiment 101, wherein said anti-CD73 antibody is capable of binding a CD73 antigen at a pH of about 6.3.

Embodiment 103. The method of any one of Embodiments 65 to 102, wherein said anti-CD73 antibody further comprises a glutamine at a position corresponding to Kabat position 297.

Embodiment 104. The method of any one of Embodiments 65 to 103, wherein said anti-CD73 antibody is bound to a CD73 antigen.

Embodiment 105. The method of Embodiment 104, wherein said CD73 antigen forms part of a cell.

Embodiment 106. The method of Embodiment 105, wherein said cell is a lymphoid cell.

Embodiment 107. The method of Embodiment 105, wherein said cell is a B cell.

Embodiment 108. The method of any one of Embodiments 65 to 107, wherein the cancer is colorectal cancer, non-small cell lung cancer, renal cell carcinoma, triple negative breast cancer, cervical cancer, ovarian cancer, pancreatic cancer, endometrial cancer, sarcoma, squamous cell carcinoma of the head and neck, bladder cancer, metastatic castration resistant prostate cancer, or non-Hodgkin lymphoma.

1. A method of activating B cells in a subject, the method comprising administering to said subject an effective amount of an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

2. The method of claim 1, wherein said B cells decrease egress from lymphoid tissue relative to a standard control.

3. The method of claim 1, wherein retention of said B cells in lymphoid organs increases relative to a standard control.

4. The method of claim 1, wherein said subject is a subject having a cancer.

5. The method of claim 1, wherein said subject is immune deficient.

6. (canceled)

7. The method of claim 1, wherein said anti-CD73 antibody is administered at a half maximal effective concentration (EC₅₀) of at least 100 nM.

8.-10. (canceled)

11. The method of claim 1, wherein said CDR L1 has a sequence of SEQ ID NO:1, said CDR L2 has a sequence of SEQ ID NO:2, said CDR L3 has a sequence of SEQ ID NO:3; said CDR H1 has a sequence of SEQ ID NO:4, said CDR H2 has a sequence of SEQ ID NO:5, and said CDR H3 has a sequence of SEQ ID NO:6.

12.-16. (canceled)

17. The method of claim 1 wherein said anti-CD73 antibody is capable of binding a CD73 antigen with an equilibrium dissociation constant (K_{D}) from about 0.3 to about 25 nM.

18.-25. (canceled)

26. An antiviral immunogenic composition comprising an antiviral immunogenic agent and an anti-CD73 antibody, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

27. The composition of claim 26, wherein said CDR L1 has a sequence of SEQ ID NO:1, said CDR L2 has a sequence of SEQ ID NO:2, said CDR L3 has a sequence of SEQ ID NO:3; said CDR H1 has a sequence of SEQ ID NO:4, said CDR H2 has a sequence of SEQ ID NO:5, and said CDR H3 has a sequence of SEQ ID NO:6.

28.-42. (canceled)

43. A method of treating cancer in a patient in need thereof, the method comprising: (i) administering to the patient an effective amount of an anti-CD73 antibody; and (ii) monitoring a level of an antigen-presenting cell.

44. The method of claim 43, wherein monitoring the level of the antigen-presenting cell comprises (a) obtaining a biological sample from the patient, and (b) detecting the level of the antigen-presenting cell in the biological sample.

45.-60. (canceled)

61. The method of claim 43, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

62. The method of claim 61, wherein said CDR L1 has a sequence of SEQ ID NO:1, said CDR L2 has a sequence of SEQ ID NO:2, said CDR L3 has a sequence of SEQ ID NO:3; said CDR H1 has a sequence of SEQ ID NO:4, said CDR H2 has a sequence of SEQ ID NO:5, and said CDR H3 has a sequence of SEQ ID NO:6.

63.-70. (canceled)

71. The method of claim 43, wherein said anti-CD73 antibody is capable of binding a CD73 antigen with an equilibrium dissociation constant (K_{D}) from about 0.3 to about 25 nM.

72.-81. (canceled)

82. A method of treating cancer in a patient in need thereof, the method comprising: (i) administering to the patient an effective amount of an anti-CD73 antibody to effectively activate an antigen-presenting cell; and (ii) monitoring a level of an antigen-presenting cell.

83. The method of claim 82, wherein monitoring the level of the antigen-presenting cell comprises (a) obtaining a biological sample from the patient, and (b) detecting the level of the antigen-presenting cell in the biological sample.

84.-99. (canceled)

100. The method of claim 82, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. 101.

101. The method of claim 100, wherein said CDR L1 has a sequence of SEQ ID NO:1, said CDR L2 has a sequence of SEQ ID NO:2, said CDR L3 has a sequence of SEQ ID NO:3; said CDR H1 has a sequence of SEQ ID NO:4, said CDR H2 has a sequence of SEQ ID NO:5, and said CDR H3 has a sequence of SEQ ID NO:6.

102.-109. (canceled)

110. The method of claim 82, wherein said anti-CD73 antibody is capable of binding a CD73 antigen with an equilibrium dissociation constant (K_{D}) from about 0.3 to about 25 nM.

111.-120. (canceled)


SEQUENCE LISTING (10 sequences)

SEQ ID NO: 1 - protein, 15 aa, Artificial Sequence
  Synthetic
RASKNVSTSGYSYMH

SEQ ID NO: 2 - protein, 7 aa, Artificial Sequence
  Synthetic
LASNLES

SEQ ID NO: 3 - protein, 9 aa, Artificial Sequence
  Synthetic
QHSRELPFT

SEQ ID NO: 4 - protein, 10 aa, Artificial Sequence
  Synthetic
GYTFTSYWIT

SEQ ID NO: 5 - protein, 14 aa, Artificial Sequence
  Synthetic
PGSGNTNYNEKFKT

SEQ ID NO: 6 - protein, 13 aa, Artificial Sequence
  Synthetic
EGGLTTEDYALDY

SEQ ID NO: 7 - protein, 120 aa, Artificial Sequence
  Synthetic
QVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTV

SEQ ID NO: 8 - protein, 111 aa, Artificial Sequence
  Synthetic
EIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIK

SEQ ID NO: 9 - protein, 471 aa, Artificial Sequence
  Synthetic
MEWSWVFLFFLSVTTGVHSQVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 238 aa, Artificial Sequence
  Synthetic
MSVPTQVLGLLLLWLTDARCEIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC